(19) United States
(12) Patent Application Publication
(10) Pub. No.: US 2009/0117633 A1
(43) Pub. Date: May 7, 2009

(54) Process of Producing Ethanol Using Starch with Enzymes Generated Through Solid State Culture

(75) Inventors: Clifford Bradley, Missoula, MT (US); Robert Kearns, Melrose, MT (US)

Correspondence Address: MORGAN, LEWIS & BOCKIUS, LLP, ONE MARKET SPEAR STREET TOWER, SAN FRANCISCO, CA, 94105, US

(73) Assignee: ENERGY ENZYMES INC., Cupertino, CA (US)

(21) Appl. No.: 12/264,875

(22) Filed: Nov. 4, 2008

Related U.S. Application Data
(60) Provisional application No. 60/985,408, filed on Nov. 5, 2007.
(60) Provisional application No. 60/985,430, filed on Nov. 5, 2007.
(60) Provisional application No. 60/985,452, filed on Nov. 5, 2007.
(60) Provisional application No. 61/021,211, filed on Jan. 15, 2008.
(60) Provisional application No. 61/024,339, filed on Jan. 29, 2008.
(60) Provisional application No. 61/097,169, filed on Sep. 15, 2008.

(51) Int. Cl. C12P 7/06; C12N 1/14; C12N 9/30

(52) U.S. Cl. 435/161; 435/256.1; 435/203

(57) ABSTRACT

The present invention is directed to process of producing ethanol using starch with enzymes generated through solid state culture.

### CROSS REFERENCE TO RELATED APPLICATIONS

This application claims the benefit of, and priority to, U.S. Provisional Patent Application Ser. Nos. 60/985,452, 60/985,430, and 60/985,408 filed on Nov. 5, 2007, 61/021,211, filed on Jan. 15, 2008, 61/024,339 filed on Jan. 29, 2008, and 61/097,169 filed on Sep. 15, 2008, the entire disclosures of both of which are hereby incorporated by reference in their entireties.

### FIELD OF THE INVENTION

The present invention is directed to process of producing ethanol using starch with enzymes generated through solid state culture.

### BACKGROUND OF THE INVENTION

One of the renewable alternative energy sources are biofuels converted from biomass. Of many of substitutes to gasoline, one of the most generally recognized substitutes which could be made available in significant quantities in the near future is alcohol, and in particular, ethanol. For example, there are currently many outlets in the United States and throughout the world which sell a blend of gasoline and about 10%-20% ethanol (commonly called “gasohol”) which can be used as a fuel in conventional automobile engines. Furthermore, ethanol can be blended with additives to produce a liquid ethanol-based fuel, with ethanol as the major component, which is suitable for operation in most types of engines. Ethanol can be produced from almost any material which either exists in the form of, or can be converted into, a fermentable sugar. There are many natural sugars available for fermentation, but carbohydrates such as starch and cellulose can be converted into fermentable sugars which then are fermented into ethanol.

Starch is one of the world's most abundant renewable raw materials. One answer to the need for alternative reproducible fuels is to convert this very abundant material at low cost into fermentable sugars as feedstock for fermentation to ethanol. A recent review article describes a long history of published research in production and characterization of raw starch hydrolysis enzymes. Robertson et al, Native or Raw Starch Digestion: A Key Step in Energy Efficient Biorefining of Grain. J. Agric. And Food Chem 54:353-365 (2006).

Conventional fuel ethanol is produced by a dry milling or wet milling process. Dry-milling starts by grinding dry corn kernels into nearly a powder, followed by mixing in water at about 30% solids, cooking (heating the starch to or above gelatinization temperatures) and treatment with high temperature alpha amylase, cooling to about 50° C. and treating with a glucoamylase to break down the starch into fermentable sugars. This sugar containing solution is cooled to 30° C., treated with yeast and fermented into ethanol via batch or continuous fermentation. The ethanol is isolated from this solution via distillation. The remaining solids in this solution are isolated and sold as cattle feed.

During wet-milling, dry corn kernels are steeped with water to allow the kernels to absorb moisture. The steep water is removed and the soaked kernels get loosely ground and processed through a number of steps to separate the germ, the fiber, the gluten, and the starch. The starch is cooked and treated with enzymes as in the dry mill process to break down the starch into sugars. Yeast are added to ferment the sugars into ethanol and purified by distillation.

After fermentation, traditional processes have removed the ethanol from the fermentation solution and further concentrated the ethanol product by distillation. Distillation towers capable of such separation and concentration are well-known in the art. Following fermentation, the 5-15% alcoholic solution, often referred to as distiller's beer or wine, is concentrated to 50-95% ethanol via distillation. This ethanol can be used “as is” to make spirits. Alternatively, the 95% ethanol, generally made at fuel ethanol plants, is passed through molecular sieves to remove the remaining water to make fuel grade ethanol, greater than 99% ethanol, used for blending with gasoline.

There is a need in the art for compositions and methods to simplify the ethanol production process from starch and to reduce the cost.

### SUMMARY OF THE INVENTION

The present invention provides a composition comprising Aspergillus phoenicis. In some embodiments, the composition further comprises a solid state fermentation substrate. In some embodiments, the substrate comprises barley. In some embodiments, the composition comprising Aspergillus phoenicis deposited as NRRL-50090.

The present invention also provides an enzyme preparation made by a solid state culture process, the process comprising: steaming a substrate to adjust moisture and reduce contamination from indigenous microorganisms, wherein said substrate comprises barley; growing a fungus on said substrate for a first period of time in a growth chamber; and harvesting said enzyme mush comprises a mixture of fungus and substrate. In some embodiments the period of time is four days. In some embodiments, the enzyme composition comprises alpha amylase. In some embodiments, the enzyme composition further comprises glucoamylase and beta glucanase. In some embodiments, enzyme composition is optimal to hydrolyze starch at pH 3.6. In some embodiments, the enzyme composition is optimal to hydrolyze starch at 20° C. In some embodiments, the steaming is conducted at ambient pressure. In some embodiments, the growing step comprises supplying nitrogen or carbon dioxide to the atmosphere of said growth chamber. In some embodiments, the substrate further comprises additional soluble nutrients. In some embodiments, the fungus is selected from a group consisting of: Aspergillus quadricinctus, A. niger, A. oryzae, A. phoenicis, A. terreus, Rhizopus arrhizus, H. delemar, R., kasanensis. R. javonicus, H. oligosporus, R. oryzae and R. thailandensis. In some embodiments, the fungus is Aspergillus phoenicis. The present invention also provides a composition comprising an enzyme preparation provided above, yeast, and mash. In some embodiments, the mash comprises un-gelatinized starch. In some embodiments, the mash comprises gelatinized starch. In some embodiments, the enzyme composition is made by a solid state culture.

The invention also provides a method for screening for a fungus, comprising: inoculating a parent fungus strain in a culture medium having a first pH value; selecting a first progeny strain that is adapted for growth at said first pH value; inoculating said first progeny strain in a culture medium having a second pH value, wherein said second pH value is lower than said first pH value; selecting a second progeny strain that is adapted for growth at said second pH value; and optionally repeating steps (c) and (d) till said second pH value is a final pH value. In some embodiments, the final pH value is about 3.6. In some embodiments, the first pH value is about 6. In some embodiments, the second pH value is 0.5 lower than said first pH value. In some embodiments, the fungus is selected from a group consisting of: Aspergillus quadricinctus, A. niger, A. oryzae, A. phoenicis, A. terreus, Rhizopus arrhizus, R. delemar, R., kasanensis. R. javonicus, R. oligosporus, R. oryzae and R. thailandensis. In some embodiments, the fungus is Aspergillus phoenicis.

The invention provides method of making enzyme composition, comprising: providing a solid fermentation substrate; growing an Aspergillus on said substrate for a first period of time in a growth chamber. In some embodiments, the method further comprises harvesting said enzyme composition. In some embodiments, the substrate comprises barley. In some embodiments, the substrate has undergone heat treatment, such as steaming. In some embodiments, the steaming is conducted at ambient pressure. In some embodiments, the substrate has undergone radiation treatment, such as gamma ray treatment. In some embodiments, the first period of time is four days. In some embodiments, the enzyme composition comprises alpha amylases, glucoamylases, and beta glucanases. In some embodiments, the enzyme composition is optimal to hydrolyze starch at pH 3.6. In some embodiments, the enzyme composition is optimal to hydrolyze starch at 35° C. In some embodiments, the substrate further comprises additional soluble nutrients. In some embodiments, the fungus is adapted for growth at pH lower than 6, lower than 4, at pH about 3.6. In some embodiments, the fungus produces at least an alpha amylase, at least a glucoamylase, and at least a beta glucanase at a pH 3.6.

The invention also provides a method of producing ethanol, comprising: providing a mash that is adjusted to pH 3.5 to 4.0; mixing said mash with an enzyme composition and yeast; and incubating for a period of fermentation time under a temperature between 20 to 40° C. to produce ethanol. In some embodiments, the method further comprises collecting and distilling the ethanol. In some embodiments, the mash has up to 40% solid. In some embodiments, the fermentation time is from 26 to 72 hours. In some embodiments, the temperature is about 35° C. In some embodiments, the mash is un-gelatinized, or gelatinized. In some embodiments, the incubating steps comprises simultaneously hydrolysis and fermentation. In some embodiments, the fungus is selected from a group consisting of. Aspergillus quadricinctus, A. niger, A. oryzae, A. phoenicis, A. terreus, Rhizopus arrhizus, R. delemar, R., kasanensis, R. javonicus, R. oligosporus, R. oryzae and R. thailandensis. In some embodiments, the ethanol produced is less than 15%.

The invention further provides a method of using the enzyme composition provided herein to produce ethanol from starch in two-step.

### BRIEF DESCRIPTION OF THE DRAWINGS

FIG. 1 depicts the scheme of using solid state culture to produce raw starch enzyme composition.

FIG. 2 depicts the comparison between conventional ethanol production process and the ATSH ethanol production process.

FIG. 3 depicts a system for producing ethanol from starch.

FIG. 4 depicts the scheme of SSC enzyme preparation, saccharification, and fermentation.

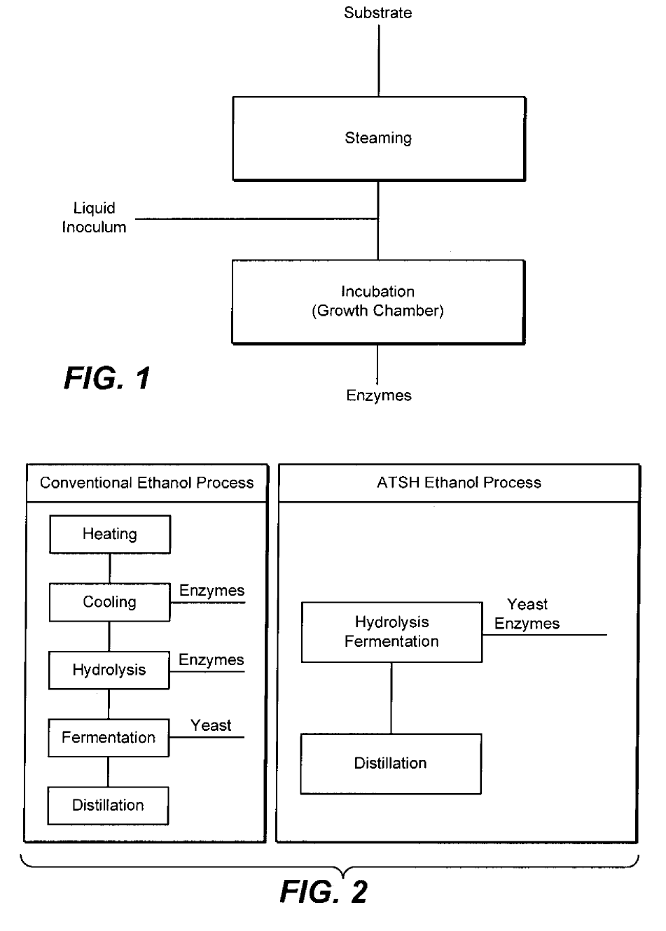

### DETAILED DESCRIPTION OF THE INVENTION

In general, the present invention relates to novel methods and compositions to produce ethanol from starch. To that end, the present invention provides methods to produce low cost enzyme preparations that contain amylase that are active against raw starch that are used to convert optionally uncooked starch into sugar, which is further fermented to produce ethanol.

In bioethanol production, enzymes are responsible for converting starch to sugars. The sugars are then fermented with yeast to make a beer containing 2-15% ethanol. One major problem in the traditional fermentation process is the contamination of undesired microorganisms. The “cooking” step in the conventional process allows both the killing of contaminating organisms and can serve as a type of pre-processing step of the starch, to “open up” the starch granules for better access to the starch enzymes. However, in some cases, additional methods for decontamination may be necessary. Current methods used to kill these unwanted microorganisms, among others, often involve introduction of foreign agents, such as antibiotics, heat, and strong chemical disinfectants, to the fermentation before or during production of ethanol. Commonly, synthetic chemical antibiotics are added to the fermentation vessels in an attempt to decrease the growth of lactic acid producing bacteria. The addition of each of these foreign agents to the process significantly adds to the time and costs of ethanol production. The use of heat requires substantial energy to heat the fermentation vessels as well as possibly requiring the use of special, pressure-rated vessels that can withstand the high temperatures and pressures generated in such heat sterilizing processes. Chemical treatments can also add to the cost of production due primarily to the cost of the chemicals themselves, and in addition these chemicals are often hazardous materials requiring special handling and environmental and safety precautions.

The present invention provides one alternative cost effective solution—to conduct the fermentation process under low pH, which will limit the growth of the contamination microorganisms, as well as the optional added benefit of the use of ambient temperature, which can significantly reduce the cost of the process.

Accordingly, the invention provides a strain of Aspergillus phoenicis (deposited with the USDA Agricultural Research Service Culture Collection, Peoria, Ill., U.S.A. as NRRL-50090), although as is outlined herein, other strains may be screened for use in the present invention. The strain is grown on a solid state substrate (sometimes referred to as “solid culture substrate” or “solid fermentation substrate” as outlined below), which optionally and preferably includes barley to induce the production of enzymes. The growth of the fungus on the substrate results in an enzyme composition that produces a variety of enzymes, optionally including amylase, glucoamylase and beta-glucanase, that have activity at low pH and ambient temperature. This is significant as the enzymatic activity at low pH allows the enzyme composition to be used on starch mashes that have undergone the traditional “cooking” step, instead relying on low pH to prevent the growth of indigenous and unwanted organisms during the starch processing.

In one aspect, the present invention provides growth substrates and growing conditions that allow production of enzyme preparations using fungus, such as the strain of Aspergillus provided herein. The invention can be generally described as follows. The substrate is selected to provide nutrition for fungal growth and the physical structure of the solid substrate culture. The dry substrate is moistened with added water or a nutrient containing solution, then steamed to adjust moisture and reduce contamination from indigenous microorganisms. The steamed substrate is cooled and inoculated with the desired fungus and loaded into a solid support growth chamber. The final moisture content of the substrate is such that the moisture is absorbed into the substrate and the substrate remains solid. The fungus grows on the substrate, utilizing it as a nutrient source, and at the same time producing the desired enzymes. This incubation time varies depending on the enzymes being produced. After the incubation, the whole culture is harvested to obtain the enzyme preparation. In many embodiments, the whole culture is used for converting starch to sugar and no additional purification of the enzymes is required. Alternatively the enzymes can be extracted and purified from the culture substrate. These enzyme preparations can be used in a process called Ambient Temperature Starch Hydrolysis (“ATSH”) that also provided herein to convert uncooked starch into sugar at ambient temperature and low pH.

Thus, the present invention provides enzyme preparations used in conversion of starch to ethanol. The selected fungal strain provide herein is grown in solid state culture to produce an enzyme preparation containing multiple enzyme activities that act on a variety of starch substrates, including raw ungelatinized starch granules, producing fermentable sugars (glucose and soluble short chain glucose polymers). The enzyme preparation can be used in multiple-step process, where the enzyme preparation is first used to convert starch to sugar, and in a second step where the sugar is fermented into ethanol; this is referred to as a “two-step process.” Alternatively, the fermentation process step can start before all starch is converted into sugar, thus there is some overlap between the starch hydrolysis step and the fermentation step. In some embodiments, as described in more detail herein, the enzyme preparation is used in a simultaneous raw starch hydrolysis and fermentation process which combines raw, ungelatinized (“uncooked”) grain mash, enzyme, and yeast in a single tank to produce ethanol.

This enzyme composition can then be added to starch mashes for starch hydrolysis, and used in conjunction with a yeast to produce ethanol.

Thus, the present invention provides two steps: an enzyme production step, and then a secondary ethanol production step optionally conducted without heating the mash to gelatinization temperatures and at low pH. Eliminating the cooking step reduces capital cost, operating cost and process energy in ethanol production.

### I. Solid Substrate Culture Technology

The instant application further provides solid substrate culture technology (sometimes referred to as solid state fermentation) to produce enzyme preparations capable of converting starch to glucose (sugar) at ambient temperatures. The use of these enzyme preparations allows production of sugar from starch without the traditional “cooking” step used in most enzymatic sugar production. Eliminating the cooking step saves capital cost and energy.

Solid culture technology has been around for over a hundred years. Most applications of solid culture technology involve the use of specific substrates or nutrients to achieve a specific end product. Sake and soy sauce are good examples. For review, see Pandey et al., Current Science (Bangalore), 77(1):149-153 (1999), herein incorporated by reference. These applications usually involve a high degree of sterilization since humans consume the resulting product. They typically use very thin layers of substrate on trays and involve a lot of material handling to prepare the substrate, grow the appropriate fungus and recover the end product. Generally food applications justify the cost of production associated with these processes.

Conventional ethanol processes uses enzymes produced in liquid fermentation. Solid substrate culture is different from conventional liquid fermentation. In a liquid fermentation system, a microorganism is placed in a liquid environment that contains soluble nutrients. Air or oxygen is bubbled through the liquid using agitation or injection to dissolve oxygen in the liquid. Generally there is not any solid support media and the oxygen available to the organism is limited by the solubility of oxygen in water. In general, these are specific enzymes that are concentrated and frequently purified to some extent.

Furthermore, when more than one enzyme is used in an ethanol process (e.g., alpha amylase and glucoamylase used in hydrolysis of starch to glucose), the individual enzymes are generally produced from different organisms grown in separate liquid fermentation vessels. One of the primary costs of enzymes produced in liquid culture is the cost of concentrating the enzymes or separating the enzymes from the broth in which they are grown. The more liquid in the process, the higher the transportation, storage, and purification costs are. The present invention provides new methods of producing enzymes at high concentrations, and generally, does not require post-production purification.

One of advantage of the SSC systems provided herein is that it mimics nature. In nature, fungi grow on moist damp surfaces, with atmospheric oxygen concentration, not in liquids. In the SSC system provided herein, when a selected fungus is grown on the proper solid nutrient source, it often produces a set of enzymes that are functionally different than the enzymes it would produce when grown in a liquid culture.

Accordingly, the instant invention provides a solid substrate culture technology that results in enzyme preparations produced from one organism with high enzyme concentrations that contains all of the enzyme activities necessary to work effectively in downstream ethanol production including from raw, uncooked starch. It should be noted that while the description herein is generally directed to processes that are run at lower pH and ambient temperatures, the enzyme preparations (or enzymes purified and/or concentrated from the enzyme preparations) also find use in traditional starch processes, or as individual enzymes for use in a wide variety of applications as is known in the art.

By “solid substrate culture (SSC)” or “solid state fermentation (SSF)” herein is meant a culture wherein the organism is grown on the surface of a moist solid material where a majority (or in some cases, all) of the water is absorbed into the substrate material. Thus, generally there is a minimum amount of or substantially no free water in the culture. As the main reason for reducing free water is to contain costs of handling and processing the enzyme preparation, it should be noted that in some embodiments, depending on the end use, there can be free water present in the culture. The substrate material provides both the nutrients and physical support for the culture. In the SSC substrate for the ATSH enzyme process described herein, water content varies from about 40 to 60% w/w depending on the actual substrate, producing a moist solid particle mix with no free water. The organism obtains oxygen from the air or from modified atmosphere introduced into the growth chamber, as is more fully described below.

In one aspect, the present invention provides process for fungal culture and enzyme preparation employing solid substrate culture. The present invention enables sufficient large scale solid substrate culture.

Furthermore, the present invention provides innovations in physical and biochemical substrate characteristics and process control that reduce costs and improve efficiency of large scale solid state culture. Substrate characteristics induce high product concentrations using low cost materials in large volume cultures, (e.g., up to ten tons of dry weight substrate in a single culture reactor). The present invention also provides methods to control temperature and moisture balance in large scale cultures with often result in very rapid generation of metabolic heat which in some cases needs to be dissipated. The selected fungal strain produces raw starch active enzymes when grown in these solid substrate cultures.

As described below, in some embodiments, the enzyme preparation provided herein comprises the whole solid substrate fungal culture including residual substrate, fungal cells and protein enzymes. When the culture reaches optimal enzyme concentration, the whole culture is harvested. The culture may be used wet without any further processing or may be dried and stored for later use. Optionally, some or all of the enzymes may be isolated from the solid substrate as well. The culture is a whole culture enzyme preparation containing multiple enzyme activities. The combination of the selected fungal strain and solid substrate culture technology produces sufficiently high enzyme titers that no further processing is required to reach usable enzyme concentrations in many embodiments. This eliminates the principal cost in producing enzymes in conventional liquid fermentation.

Furthermore, exogenous starch active enzymes (for example from different fungus) can also be added to the enzyme preparation, as is further described below.

In another aspect of the present invention, the enzyme preparation provided herein can be further purified, or partially purified, to produce enzymes with higher purity or activities. It can also be used to purify specific enzymes with enzyme purification technologies known in the art.

### A. Substrate Selection and Preparation

The process of producing enzymes by growing a fungus on solid culture begins with selection of the proper fungus and substrate. The selected fungi should be able to metabolize starch.

There are known methods of growing fungus on solid substrate; see for example, Ellaiah P. et al. Process Biochemistry, 38(4):615-620 (2002); U.S. Pat. No. 6,558,943, incorporated by reference herein in its entirety.

The present invention provides solid state culture substrates with moisture retention capability and physical strength to use in a packed bed without collapsing or “mushing down”. These solid substrate culture substrates are processed to provide a material with both the physical and nutritional requirements necessary for optimal fungal growth and enzyme production. Some additional soluble nutrients are optionally added to achieve the desired fungal growth and enzyme complex.

Many different solid substrates can be used for the production of enzymes using fungus, such the production of glucoamylase employing Aspergillus under solid state fermentation. These include, but are not limited to, wheat bran, green gram bran, black gram bran, corn flour, barley flour, jowar flour, maize bran, rice and rice bran. Substrates are generally moistened with water and steam sterilized or otherwise treated to reduce competing indigenous micro-organisms.

In general, the substrate comprises a mixture of components.

In one embodiment, a component of the substrate used for the ATSH amylase production is barley. Barley (Hordeum vulgare) is a cereal grain, which serves as a major animal feed crop, with smaller amounts used for malting and in health food. Barley not only acts as source of nutrition for the fungus, but also appears to act as an inducer for amylase production. Experimental evidence suggests that short chain soluble beta 1,3 linked glucans act as very powerful inducers of amylase activities.

In one embodiment, the substrate comprises barley, primarily steam rolled or hulled barley. To form the substrate, one embodiment utilizes steam rolled or hulled barley, which is wetted with a nutrient solution and steamed prior to inoculation as described below. Other forms of barley may be used as well

In another embodiment, finely ground barley is mixed with a nutrient solution (at between 20 and 60% moisture) and extruded to form pellets which comprise the solid culture substrate. The extrusion process creates high temperature and pressure as the barley/water is forced through the extruder die so that steaming the substrate to control contamination is not necessary.

By “rolling” or “grinding” herein is meant the processes used to reduce the size of whole grains. The physical forces employed include, but is not limited to, impact to create fractures, abrasion/attrition to scrape off material, shear to slice apart, and pressure to crush (deform) structure. Particle size reduction is important to solid state culture for the following reasons. First, particle size reduction increases surface area, which leads to improved utilization of grains through increased exposure of endosperm material to fungus. Second, particle size reduction provides improved mixing characteristics of dissimilar substrate ingredients. Third, particle size reduction provides improved handling of fibrous feedstuffs.

By “steamed rolled” herein is meant the process in which steam is applied to barley before rolling, Steam rolled barley is produced by exposing barley to steam for three to five minutes and then rolling it. This process produces fewer fines than dry rolling or grinding. Steamed rolled barley is commonly used as feedstock in the cattle industry to increase Feed consumption and weight gain.

By “hulled” herein is meant the outer hull is removed from barley. The separation of outer hull from inner barley groat can be done by methods of centrifugal force. Barley grains can be gravity fed on to the center of a horizontally spinning stone to be thrown to the out ring where the oat and hull will separate from to the impact. The lighter barley hulls will be then aspirated away while the denser barley groats will be taken to the next step of processing.

The extent of rolling can be controlled. Typically the rolled barley can be used in the present invention is from commercial feed mill and is just a flattened barley kernel maybe 2 to 4 mm thick. Finely ground barley used to make the extruded pellets is barley ground in a hammer mill or other mill to produce a powder or flour that passes a 20 mesh US standard screen. The barley may be whole grain or may be hulled prior to milling.

The percentage of barley in the total substrate can be from 10 to 99% (w/w), preferably from 50 to 90%, and even more preferably from 80 to 90% Typically dry steam rolled or dehulled and steam rolled barley is mixed with water/nutrient solution to about 40 to 50% moisture content, (equal weight of barley and solution gives a 50% moisture content). The solid substrate can also include straw. The function of the straw is to open up the culture bed structure to facilitate aeration. In some embodiments, the substrates comprise straw in pieces of 0.5 to 3 cm long at rate of about 1 to 5% w/w on a dry basis.

As will be appreciated by those in the art, many different combinations of substrates can be used including those outlined herein for fermentation. The description herein is meant to include all possible combinations of substrates, including combinations that lack particular components.

In addition to the grain components, the invention utilizes a wetting solution. In one embodiment, this solution can be water, which is sufficient for fungal growth and enzyme production on substrates, including barley substrates, although in general, higher titers of enzymes are produced when a nutrient wetting solution is used. Thus the term “wetting solution” includes both water as well as solutions containing additional nutrients and/or chemicals such as acid to control pH.

Thus, nutrient wetting solutions find use in the present invention in many applications. The nutrient solution can add additional nutrients in a variety of forms for use by the fungus, as well as be used to adjust the pH of the substrate, For example, nutrient solutions can contain nitrogen sources, acids and bases or buffers, and minerals. Nutrient wetting solutions of particular use include solutions containing urea, ammonium phosphate and sulfuric acid. A particular nutrient solution contains urea at 16 grams per liter, ammonium phosphate at 13.3 grams per liter, and sulfuric acid at 13.3 ml one molar solution per liter of water. In some embodiments, stillage is added as nutrient wetting solution as described herein. In some embodiments molasses solution of 1 to 10% is added as a nutrient wetting solution.

In general, the wetting solution is added to the substrate to result in a desired final moisture content, although in some cases additional water or nutrient solution can be added periodically to the fungal fermentation as well. For example, if steaming of the substrate is used to reduce bacterial contamination is used, as is described below, this step generally introduces additional water. Thus, the final moisture content of the substrate, as well as the moisture content that is maintained during the enzyme production, can be reached using nutrient solutions and/or water.

The substrate components used herein can be processed or raw agriculture products. Raw agricultural products frequently have indigenous microbial contamination. Left untreated, these contaminants will compete with the slower growing fungus, and potentially out-compete the desired fungi, resulting in a contaminated product, low quality product, or no useable product. As is known in the art, there may be a variety of techniques used to reduce the contamination, including, but is not limited to, heating (including steaming), radiation, and treatment with antibiotics. In some embodiments, a steaming process is employed to handle large quantities of solid materials.

Steaming finds particularly use in the present invention. By “steaming” herein is meant the process that applying vaporized water to a material, such as the substrate for solid state culture described herein. Steaming is one of the common methods of sterilization for the elimination of microorganisms such as bacteria. Water vaporizes when heated to 10° C. under standard atmosphere pressure (100 kPa). However, under higher pressure, water will only vaporize at temperature higher than 100° C. Thus steaming can be carried out at ambient pressure, such as atmosphere pressure, without extra pressure being applied. Alternatively, steaming can be carried out under pressure higher than 100 kPa. Steaming carried out under pressure higher than 100 kPa is called autoclaving. Autoclaves commonly use steam heated to 121° C. (250° F.), at 103 kPa (15 psi) above atmospheric pressure. Solid surfaces are effectively sterilized when heated this temperature for at least 15 minutes or to 134° C. for a minimum of 3 minutes. “Effective sterilization” in this context includes methods to reduce undesired microorganisms, such as bacteria, to the extent that they can not interfere with the enzyme production process.

With the Aspergillus amylase solid substrate culture process described herein, steaming at ambient pressure is sufficient; that is, pressure sterilization of the substrate is not necessary (although it can find use in some processes).

By “ambient pressure” herein is meant a pressure that is close to the atmosphere pressure in a given site. The atmosphere pressure changes according to the altitude and latitude, and can be measured by standard atmosphere (1 atmosphere=101.325 kPa). Thus for most locations, the ambient temperature is about 100 kPa.

By “ambient temperature” here is meant a temperature that is between 15-50° C., and preferably is between 18-40° C., and more preferably the temperature is 35° C.

By “Standard ambient pressure and temperature” herein is meant 25° C., 100 kPa.

Steaming can also optionally be used to adjust the amount of water in the substrate. A certain amount of water is necessary for the growth of the fungus. Water can be added to the substrate together with other components. However, because there is only a limited amount of water needed for making the substrate, it may be difficult to mix the water evenly in the substrate. Thus, steaming, among other techniques, such as sprinkling during mixing, is a convenient way to introduce water to the substrate evenly. Substrate moisture after steaming may be in the range of 30 to 80% preferably 40 to 50% in barley substrates.

If other decontamination techniques are used, water may need to be introduced separately. If the substrate is extruded, for example using barley pellets, ground barley is mixed with water or nutrient solution to a 30 to 60% moisture content to form a moist solid dough which is forced through the extruder die at any combination of pressure and temperature sufficient to form a moist solid pellet. The preferred pressure may range from 50 to 300 psi and temperature from 50 to 150 degrees C. Any equipment capable of forming extruded pellets by forcing material through a die may be employed.

Thus, in one aspect of the present invention, the substrate is steamed to adjust moisture and reduce contamination from indigenous microorganisms. In one embodiment, the substrate is steamed by applying steam. This can be carried in open space, where the substrate is spread out on a surface, such as the floor, or the bottom of a container. However, preferably, steaming is carried out in a contained space, such as a growth chamber, and optionally, mechanical methods is used to mix and move the substrate to assist in the even distribution of steam throughout the substrate. Steaming can carried out under pressure higher than atmosphere pressure when steam is introduced into a closed, pressured system. Alternatively, steaming is carried out at ambient pressure, such as the same as the atmosphere pressure, where the steam is introduced into an open system.

The duration of the steaming depends on the amount and density of the substrate. It can be from several minutes to several hours, preferably from 10 to 30 minutes up to 4 hours. Substrate may also be double steamed in a process called tyndalization. In this process the substrate is steamed for a period preferably 1 to 30 minutes, then allowed to cool to about 3° C. and held for a period of 4 to 24 hours, preferably about 12 hours. The substrate is then steamed again for a period of 10 to 30 minutes.

After steaming, the substrate will be let cooled down to a temperature suitable for the growth of fungus, either by naturally cooling down over time, or by applying cold air to the substrate.

Final moisture after addition of a liquid inoculum culture is preferably in the range of 45 to 55%. Final moisture content of the substrate is determined by the absorbency or water holding capacity of different substrate materials under different process conditions of temperature a pressure. With barley substrates final moisture content can range from the minimum water activity at which the selected fungal strain will grow, about 30% moisture in barley substrates, to a maximum at which the substrate is no longer solid, in barley substrates about 80% final moisture. Final moisture refers to moisture content of the substrate after nutrient solution or water addition, steaming and inoculation. Then the components are mixed together, preferably by a mechanical method.

Optionally, the pH of the substrate is also adjusted to low pH. The pH can be from 3 to 7, preferably from 3.5 to 5. As is known in the art, many different chemicals can be used to adjust the pH, such acid, including, but is not limited to, ammonia, sulfuric acid, phosphoric acid acetic acid, lactic acid, citric acid, and hydrochloric acid. The mixing of the substrate and the adjusting of the pH can be carried out in a single step, or in separate steps.

As used herein, the term “about” modifying any amount refers to the variation in that amount encountered in real world conditions of producing sugars and ethanol, e.g., in the lab, pilot plant, or production facility. For example, an amount of an ingredient employed in a mixture when modified by “about” includes the variation and degree of care typically employed in measuring in an ethanol production plant or lab. For example, the amount of a component of a product when modified by “about” includes the variation between batches in an ethanol production plant or lab and the variation inherent in the analytical method. Whether or not modified by “about,” the amounts include equivalents to those amounts. Any quantity stated herein and modified by “about” can also be employed in the present invention as the amount not modified by “about.”

The substrate then can be used to grow fungus.

### B. Fungal Inoculum Preparation, Incubation and Culture Control

The steamed substrate is inoculated with the desired fungus (the inoculum) and loaded into a growth chamber. The fungus grows on the substrate, utilizing it as a food source and at the same time, producing the desired enzymes.

By “inoculum” or “inoculant” herein is meant the material used in an inoculation. For example, the fungus provided in the present invention, or the fungus that are obtained through the methods provided in the present invention, or any other suitable fungus, is produced in conventional liquid culture known in the art to produce a large volume of cell mass. These cells are sprayed on the steamed substrate as an inoculum.

The methods to produce inoculum are well known in the art. Generally, fungus from a stock can be used to grow on a medium, either liquid or solid, for a period of time under proper temperature. The temperature is 20-45° C., preferably 20° C.-35° C., and more preferably 30° C. The incubation time is one day to one month, preferably 2 to 20 days, more preferably 2 to 15 days, and even more preferably 1 to 5 days. After the fungus reach the desired density in the liquid culture, or desired colony size on the surface of solid culture media, they are harvested to be used to inoculate substrate in a growth chamber. Either the fungal cells, the spores formed by the fungi, or mixture of both, can be used as inoculum. Spores can be harvested by methods known in the art, for example, by washing the surface of an agar plate or on a solid culture substrate of smaller volume than the production culture on which fungi grown with either water or buffer, and separate spores by known methods, such as filtering and centrifugation. Spores are easy to store and have a much longer shelf life.

Among the factors that determine morphology and the general course of fungal fermentations, the type and size of inoculum is of prime importance. By “inoculum size” herein is meant the amount of inoculum being used for the inoculation. It is measured by the percentage of inoculum weight over the substrate weight. Suitable type and size of inoculum can be determined using methods known in the art. For example, different inoculum size, such as from 0.1-20% (w/w), can be tested in small scale fermentor to determine the optimal size. Inoculum size can be from 0.1-20%, preferably from 0.5 to 5%, and even more preferably from 1-2%, and even more favorably is about 2% (w/w). In one embodiment, the selected fungal strain is grown on a solid culture substrate such as the barley substrate described above until the fungus produces spores. The spore culture is then dried and stored. The spore culture material is used to inoculate the solid culture substrate such that the ratio of spores to substrate is in the range of 100 to 100 million spores per gram of substrate, preferably about one million spores per gram of substrate. As will be appreciated in the art, the size of the inoculum can range depending on the desired time of growth.

The fungus provided herein can be grown in a liquid medium know in the art. In some embodiments, the A. phoenicis is grown in a liquid media consisting of 5% molasses and 1% ammonium phosphate. In other embodiments, A. phoenicis is grown in a YM broth, a standard laboratory media containing yeast extract, malt extract and glucose.

In one aspect, the present invention provides methods of incubating and growing fungi in a growth chamber or bioreactor using solid state culture technique.

By “growth chamber” or “bioreactor” herein is meant any device or system that supports a biologically active environment, particularly a device capable of holding fermentation media inoculated with microorganism and carrying out the process of solid state fermentation in a contained manner. Bioreactors are commonly cylindrical, ranging in size from some liter to cubic meters, and are often made of stainless steel. A growth chamber can be used to grow any microorganism capable of growing under specified conditions in a contained environment. It can be equipped to control the temperature, pH, air composition, humidity, intensity of light, etc. within the device to provide a desired environment for the microorganism to grow. In some embodiments, the growth chambers are rectangular in shape and constructed of mild steel or plastic panels designed for ease in cleaning. For example growth chambers designed for commercial use might have dimensions of 10 feet wide, 10 feet high and 60 feet long with a series of trays or shelves stacked at 6 inch to one foot intervals. Shelves are constructed of mesh material to allow air circulation to and from the bottom.

Under optimum conditions the microorganisms or cells are able to perform their desired function with great efficiency. In solid substrate culture, the culture substrate with the growing fungi may be static or agitated. In many embodiments designed for amylase production using Aspergillus as described herein, culture beds are static and do not require mixing, although mixing finds use in certain applications. The bioreactor's environmental conditions including gas (i.e., air, oxygen, nitrogen, carbon dioxide) compositions and flow rates, temperature, pH and relative humidity can be closely monitored and controlled. The growth substrate temperature and growth conditions are also monitored and controlled by changing the growth chamber environmental conditions as needed and described herein

In solid culture, the fungus grows on the surface of and penetrates into the moist solid substrate particles. Fungal cells are directly exposed to atmospheric oxygen. Dissolved oxygen and the aeration agitation necessary in liquid culture is generally not relevant to the solid culture system. In solid culture systems reported in the literature temperature is controlled by air flow through the culture substrate and or by mechanical systems such as temperature controlled trays or heat exchangers in the culture bed. In some embodiments, air flow and air temperature are used as one method of temperature control. However a very important innovation in the system of the present invention is manipulation of the gas composition of the atmosphere to control the rate of metabolism and of metabolic heat generation. Specifically the present invention allows cultures to deplete oxygen and or enrich the atmosphere with carbon dioxide or nitrogen in response to culture temperature. This slows metabolic rate and reduces heat generation during periods of peak metabolic rate in the culture.

Specific designs for solid substrate culture equipments are described, for example, in U.S. Pat. Nos. 6,197,573, 6,664,095, and 6,620,614, herein all incorporated by reference.

Controlling the temperature of large quantities of rapidly growing fungal culture is preferred in some embodiments. If not controlled or removed, metabolic heat generation will increase culture bed temperature to the point where fungal growth is inhibited.

The present invention provides processes to both control metabolic rates and efficiently remove heat while maintaining substrate moisture. The packed substrate bed is designed to allow air circulation and heat removal. Control of bed moisture and air humidity is an important factor in the success of solid substrate systems. Air circulation will tend to dry the substrate, which can reduce the amount of waterbelow the point where fungi will grow, unless additional wetting solution is added. Temperature control for the Aspergillus SSC is also key, as the A. phonencis strain grows very rapidly in certain systems, which can generate very high peak heat loads.

Thus, the present invention includes controlling the metabolic rate of growth of the fungus. A suitable control process employed with Aspergillus cultures monitors and controls the oxygen content in the culture atmosphere to control the metabolic rate of the fungus. By using carbon dioxide from the substrate metabolism supplemented by controlled additions of either nitrogen or carbon dioxide to the atmosphere in the SSC chamber the present invention provides a process that can control the rate of metabolic heat generation and manage peak heat loads in the culture without affecting the titer or composition of the enzyme complex. Typically oxygen concentration in the culture is monitored and controlled between about 2% and about 5% to reduce peak metabolic heat generation and prevent peak metabolic heat generation from increasing culture substrate temperature above 36 degrees C. At about 5% oxygen concentration metabolic heat generation is slowed sufficiently to prevent a rise in culture temperature. At the 2% oxygen, metabolic rate is generally slowed sufficiently to reduce culture temperature if necessary. In addition, maintaining oxygen concentration at 5% or less prevents spore formation by the Aspergillus strain (or by other fungal strains tested). Preventing spore formation brings multiple advantages to SSC: exposure to spores may cause allergic response in sensitive individuals; airborne spores are difficult to control and may contaminate other cultures or processes conducted in a facility; and spore color in final enzyme preparations might cause problems with customer acceptance. While the presence or absence of spores makes no difference to the composition of final enzyme preparation, users may find the black color imparted by the Aspergillus spores undesirable; without spores the whole culture enzyme preparations are a brownish color common in the industry.

The air composition within the chamber is also important. Fungus grows under aerobic conditions, thus a sufficient supply of oxygen is important. Carbon dioxide is generated by the fungus, thus should be remove from the chamber from time to time to prevent the inhibition of fungus growth. Thus a good air circulation system finds use in the invention. Thus fresh air can be introduced into the chamber to replace the air therein. Generally, air from the atmosphere contains 78% nitrogen, 20.95% oxygen, 0.04% carbon dioxide, and about 1% water vapor.

In some embodiments, for optimal fungus growth at the beginning of the growth period, the growth chamber atmosphere is maintained with normal fresh air with 20.1% oxygen and about 0.04% carbon dioxide. To control metabolic heat generation and aid in temperature control, introduction of fresh air is limited to allow oxygen to be depleted and CO₂ to increase. In addition nitrogen or CO₂ may be introduced into the air circulation to reduce oxygen concentration. During peak metabolic period, oxygen is maintained between 2% and 5% to reduce metabolic heat generation and aid in temperature control. Fresh air may be introduced again to maintain culture growth.

The humidity inside the growth chamber is also controlled. Generally, humidity is measured in term of relative humidity (“RH”), which is defined as the ratio of the partial pressure of water vapor in a gaseous mixture of air and water to the saturated vapor pressure of water at a given temperature. The RH inside the chamber is 10-90%, preferably 80 to 90%, and even more preferably 90%.

Fungi can secrete metabolites that may change the pH of the substrate during the course of incubation. Thus, it may necessary to adjust the pH during the course of incubation. Generally, the pH inside the growth chamber is 3-6, preferably 3-5, and more preferably is about 3.5. Acid is added to the substrate along with other nutrients to reduce the pH to about 4. Generally, pH is not adjusted during culture incubation

The process uses technology innovations adapted from the malting and mushroom industries. The mechanics of being able to move large quantities of solid substrate, mix and maintain uniform moisture, and uniformly heat and cool the beds is known in the art. In one aspect, a mechanical method is employed to move and mix the substrate within the growth chamber. The mechanical methods can be blades that can blend the substrate, or a shaking device on top of which the substrate is placed. Any mechanical system to efficiently mix a solid material such as barley substrate with water solutions is suitable for the present invention. In one system, dry substrate in a “mixing chamber” is stirred by the action of hollow flight agars set vertically that lift the substrate up through the center of the agar where it falls out the top providing vertical mixing. While turning, the agars travel horizontally through the substrate to mix the entire substrate bed. Water nutrient solution and or steam may be added while the substrate mixes. Alternatively substrate can be mixed and wetted using paddle mixers of standard commercial design. Water, nutrient solution or steam can be added to the substrate during operation of the paddle mixer. After wetting and steaming, substrates (for example composed of steam rolled barley or barley flakes) can be inoculated and transferred into the growth chamber using conveyor systems. In another system, finely ground barley mixed with a water solution in a paddle mixer or other mixing device is fed through an extruder to form substrate pellets. The pellets are inoculated and loaded into the growth chamber. The growth chamber also includes at least a gas inlet and a gas outlet to allow the circulation of the air inside the growth chamber. The air introduced into the chamber is preferably pre-cleaned, such as by filtering, to remove undesired contaminants, particularly bacteria. The source of the air can be from atmosphere, or from a gas tank. The air can be mixed with other gases in the growth chamber including but not limited to oxygen, nitrogen, and carbon dioxide, all of which may be generated on-site. Alternatively, gas, such as oxygen, nitrogen and carbon dioxide can be injected separately, pre-mixed with air or each other, and injected into the growth chamber through a separate inlet. Steam can also be introduced into the growth chamber if desired to maintain the humidity inside the growth chamber. The outlet is optionally connected to a cleaning method, such as a filter or scrubber, to prevent the spores in the released air from reaching the environment. Such spores, especially spores from a fast growing fungus, may contaminate other facilities nearby and affect the production.

The growth chamber preferably is also attached to a variety of sensors to monitor the conditions, such as temperature, humidity, pressure, air composition, and pH within the chamber. A variety of sensors are known in the art and can be used to monitor the conditions with the growth chamber.

The growth chamber is also preferably attached to control methods that can control the conditions, such as temperature, humidity, pressure, air composition, within the chamber.

In one embodiment, a Programmable Logic Controller®, PLC®, or Programmable Controller is used to control the reactor. A programmable controller is an electronic device used for automation of industrial processes, such as control of machinery on factory assembly lines. Unlike general-purpose computers, the PLC is designed for multiple inputs and output arrangements, extended temperature ranges, immunity to electrical noise, and resistance to vibration and impact. Programs to control machine operation are typically stored in battery-backed or non-volatile memory. A PLC is a real time system where output result is produced in response to input conditions within a bounded time.

PLC generally has extensive input/output (I/O) arrangements. These connect the PLC to sensors and actuators. PLCs read limit switches, analog process variables (such as temperature and pressure), and the positions of complex positioning systems. On the actuator side, PLCs operate electric motors, pneumatic or hydraulic cylinders, magnetic relays or solenoids, or analog outputs. The input/output arrangements may be built into a PLC, or the PLC may have external I/O modules attached to a computer network that plugs into the PLC. PLCs may also have a human-machine interface to interact with people for the purpose of configuration, alarm reporting or everyday control.

In some embodiments, the present invention uses a process control program and PC to monitor feedback loops to control Solid Substrate Culture incubation of the fungi. The computer controls electronically actuated valves opened or closed to provide: outside air (or tank gas flow) to responding to temperature measurement and oxygen concentration in chamber atmosphere to control oxygen level; and steam injection into the air flow in response to humidity measurement. Control systems may also divert air flow through heaters or refrigeration to heat or cool the air circulating through the growth chamber.

One property for monitoring and control is temperature. Controlling the temperature of large quantities of rapidly growing fungal culture is preferred in some embodiments. The growth rate of fungus can depend on the temperature. In general, the growth of fungus is a heat generating process, cooling is more likely to be used than heating. If not controlled or removed, metabolic heat generation can increase culture bed temperature to the point where fungal growth is inhibited.

The control of temperature is by transfer of heat in or out of the growth chamber, thus heating or cooling the temperature inside the growth chamber. There are a variety of methods to transfer heat and control the temperature. In some embodiments, the control of the temperature is by circulation of air. For example, circulation of air inside the growth chamber can be coupled with the exchange of the air between the inside and the outside of the chamber. Alternatively, hot or cold air can be blown into the chamber if desired.

In some embodiments, a thermal jacket can be attached to the outside of the chamber, with heat carrying media inside the thermal jacket. The heat carrying media can be solid material or aqueous liquid, such as water, circulation in it. The liquid can be cold or warm, depends on whether cooling or heating is desired. The thermal jacket can be connected to a heating or cooling device. Alternatively, the thermal jacket can comprise a cooling or heating device itself. In some embodiment, the thermal jacket comprises an electric heater.

In many embodiments, a constant or substantially constant temperature is maintained inside the growth chamber. This can be accomplished by methods such as agitation of the substrate.

In some embodiment, the transfer of heat between the growth chamber and outside is combined with the agitation of the substrate inside the chamber to maintain a substantially constant temperature inside the growth chamber.

The temperature in the solid culture substrate in the growth chamber should be controlled for optimal fungus production. It is between 10-50° C., and preferably is between 25-45° C., more preferably between 32-40° C., and even more preferably is about 36° C.

Usually, during the incubation, the fungus metabolizes the substrate, and generates heat (and enzymes), therefore there is some waste heat to dispose of. It is a low value heat, typically less than 30° C. It can be used as supplemental room heat or exhausted to atmosphere.

The temperature inside the chamber is generally warmer than the atmosphere at the site. However, it may be desirable to have growth chamber pressure lower or higher than the atmosphere at the site. For example, the pressure inside the chamber may be lower than outside in order to prevent the spores produced during the incubation from escaping to contaminate the environment. Conversely, the pressure inside the chamber may be higher than outside to prevent microorganisms, such as bacteria, from entering the growth chamber. Growth chambers usually operate under positive pressure relative to the outside atmosphere.

The growth chamber generally also includes at least an air inlet and an air outlet to allow the circulation of air (or gas) inside the growth chamber. The air come into the chamber is preferably pre-cleaned, such as by filtering, to remove undesired contaminants, particularly bacteria. The source of the air can be from the atmosphere. The air can be mixed with other gases in the growth chamber including, but is not limited to oxygen, nitrogen, and carbon dioxide, all of which may be generated on-site. Alternatively gas, such as oxygen, nitrogen and carbon dioxide can be injected separately, pre-mixed with air or each other, and injected into the growth chamber through a separate inlet. Steam can also be introduced into the growth chamber if desired to maintain the humidity inside the growth chamber. The outlet is preferably connected to a cleaning method, such as a filter or scrubber, to prevent the spores in the released air from reaching the environment. Such spores, esp. spores from a fast growing fungus, may contaminate other facilities nearby and affect the production.

The pressure inside the chamber is generally higher than the atmosphere at the site. However, it may be desirable to have pressures lower or higher the atmosphere at the site. For example, the pressure inside the chamber may be lower than outside in order to prevent the spores produced during the incubation from escaping to contaminate the environment. Conversely, the pressure inside the chamber may be higher than outside to prevent microorganisms, such as bacteria, from entering the growth chamber. Generally, the growth chambers run at small positive pressure, measured in inches of pressure. The spores are not easily airborne because they are wet, and there is a static bond and sometimes physical bond to the growth substrate.

For optimal fungus growth, generally, the oxygen within the chamber is from about 1 to about 30%, preferably from about 2 to about 21%, and during periods of peak metabolism from 2 to 5%. Generally, the oxygen concentration starts of at atmospheric concentrations, it could be higher but that may add an unnecessary cost and doesn't necessarily increase metabolism. The oxygen concentration can be anything above 5% without inhibiting growth and/or enzyme production for the first 12-18 hours. After 12 hours the oxygen concentration generally drops to 5-9%. After 18 hours (the onset of peak metabolism), the oxygen concentration generally drops to 2-5%. The fungus will grow at lower oxygen (less than 1%) but the growth becomes inconsistent through the bed, affecting the consistency of enzyme production. At oxygen concentrations above 5%, it is generally necessary to employ mechanical means to remove heat, i.e. refrigeration, agitation, etc. The concentration of carbon dioxide and/or nitrogen will increase proportionally as the oxygen concentration decreases. The concentration of carbon dioxide is from 0.04 to 98% (w/w), preferably from 0.4 to 19%, and even more preferably during and after peak metabolism from 17 to 19% w/w.

The humidity inside the growth chamber is also controlled. Generally, humidity is measured in term of relative humidity (“RH”), which is defined as the ratio of the partial pressure of water vapor in a gaseous mixture of air and water to the saturated vapor pressure of water at a given temperature. The RH inside the chamber is from 10 to 100 (w/w), preferably from 50 to 95%, and even more preferably from 90 to 95%. Generally, the relative humidity will be whatever atmosphere RH is at the time the chamber is loaded (e.g., typically 10-15% in Montana). As soon as it is loaded, the chamber is closed and the humidity level is raised as high as possible (95% is the general limit on measuring humidity) and maintained as high as possible during the entire growth cycle. This is done to help prevent the substrate from drying out.

The incubation time varies depending on the enzymes being produced. It is from 2 to 15 days, preferably 3 to 7 days, and more preferably about four days for growing Aspergillus for ATSH. After the incubation, the whole culture is harvested for the next step in the process.

In one embodiment, the fungi metabolize approximately 45% to 55% of the substrate during incubation. It can exit the growth chamber at about 50% moisture. On a dry basis, for each 100 lbs of substrate input, 45 to 55 lbs of enzyme preparation is recovered.

### C. Enzyme Preparation

There are commercially available purified enzymes for starch hydrolysis. For example, alpha-amylase and glucoamylase preparations are marketed by Genencor International Inc. (SPEZYME® series of thermostable alpha-amylase and DISTILLASE® series of glucoamylase) and by Novozymes Inc.

The present invention provides a raw starch enzyme preparation and methods of making the enzyme preparation. The usage of the enzyme preparation as provided in the present invention provides significant cost-reduction in producing ethanol from starch.

By “enzyme preparation” herein is meant the composition containing a mixture of enzymes that efficiently hydrolyze starch in raw ungelatinized starch granules at low pH and ambient temperature.

By “low pH” herein is meant the pH from 3.5 to 5.5, preferably from 3.5 to 4.5.

In many embodiments, the whole culture is used as an enzyme preparation without any purification steps. This way, the cost of producing enzyme preparation can be dramatically reduced. Accordingly, the whole culture is mixed in water and pumped to an ethanol fermentation tank or dried and stored for future use. Since the whole culture is used as the enzyme preparation, there is no waste product to dispose of.

If the enzyme preparation is to be used directly in the ethanol production process, water can be added to the whole culture to make a slurry. The amount of water to be added depends on the characteristics of the pump. Typically the slurry will contain 30%-50% w/w solids.

The enzyme preparation can be used in the ethanol production methods provided herein or any other suitable process known in the art.

Alternatively, the whole culture can be dried for storage using methods known in the art. The whole culture can be air dried at temperatures below 36° C., a freeze drier can be used, or a vacuum dryer.

In some embodiments, the whole culture is harvested for purifying enzymes that can be used to convert starch to sugar. The purification can be carried out according methods known in the art to separate the enzyme proteins from the culture substrate, for example by extracting the culture in water or buffer solution, then concentrating the resulting enzyme containing solution or by using known chromatography techniques to purify the enzyme proteins. The purification can be complete or partial, and can include just removing the remaining solids and fungal cells or higher levels of purification as outlined herein, including diafiltration, ultrafiltration, and chromatography.

Generally, the fungi are harvested and separated from the culture media by methods known in the art, such as centrifugation. Generally, secreted amylases are recovered from the liquid cultures as follows: The culture supernatant was adjusted to 20% saturated ammonium sulfate and stirred for one hr. at 4° C. After centrifugation, the resultant supernatant was adjusted to 70% saturated ammonium sulfate and stirred for one hr at 4° C. After centrifugation of the supernatant, the resultant pellet was re-dissolved in 50 mM sodium acetate, pH 6.0, 5 mM calcium chloride, and sterile filtered. When SSC is used, enzymes can be recovered by washing the culture with a cold buffer (such as PBS) before being adjusted to 20% saturated ammonium sulfate as above.

To prevent enzyme degradation and denaturing, the purification is preferably carried at low temperature, such as at 4° C., and in the presence proteinase inhibitors. There are many proteinase inhibitors known in the art and are commercially available.

In another embodiment, the whole culture is used as an enzyme preparation without any purification steps. This way, the cost of producing enzyme preparation is dramatically reduced. Accordingly, the whole culture is slurried and pumped to an ethanol fermentation tank or dried and stored for future use. Since the whole culture is used as the enzyme preparation, there is no waste product to dispose of.

If the enzyme preparation is to be used directly in the ethanol production process, water can be added to the whole culture to make a slurry. The amount of water to be added depend on the type of pump used to transfer the slurry to the fermentation tank. Typically the slurry would contain about 30-50% dry weight whole culture solids.

Alternatively, the whole culture can dried for storage using methods known in the art. The whole culture can be dried by any method in which the temperature of the culture substrate does not exceed 36° C. during the drying process. In some embodiments, warm dry air is circulated around and through the substrate. During initial stages of drying, air temperature may exceed 50° C. as evaporative cooling maintains substrate temperature below 36° C. As the culture dries air temperature is reduced.

The amount of ATSH enzyme preparation required for starch hydrolysis depends on the enzyme activities of the enzyme preparation as well as the nature of the feed stock that provide the source of the starch. Thus, the enzyme activities of the enzyme preparation and the content of the starch of the feedstock can be measured using methods known in the art of those described herein. Small scale pilot runs can also be conducted to determine the optimal amount of enzyme preparation needed. Enzyme activities are determined as discussed below.

In one embodiment, the ATSH enzyme preparation is typically added to the ethanol fermentation on the basis of a ratio of total weight of dry weight equivalent whole culture to the dry weight equivalent of the ethanol substrate. This ratio may be in the range of 0.125% to 20% of whole culture to ethanol feedstock or 1.25 to 200 grams dry weight whole culture to each 1000 grams of whole ground starch containing ethanol feedstock. The preferred process for ethanol production from raw starch uses a simultaneous starch hydrolysis and fermentation in which ground grain (or other starch containing ethanol feedstock) is mixed with water to form a mash, acid added to adjust pH and to which enzyme and yeast are added such that the glucose from the enzymatic hydrolysis of the raw starch is immediately converted to ethanol by the yeast. In this process the overall rate of ethanol production is determined by the rate of raw starch hydrolysis which is determined by the ratio of enzyme to raw starch—the rate of raw starch hydrolysis. In some embodiments enzyme to feedstock ratio is such that the complete conversion of starch to ethanol takes place over a time period with a final ethanol concentration typical of the conventional fermentation process. This is generally in the range of 0.25% to 5% enzyme to ethanol feedstock to achieve a final ethanol concentration in the fermented “beer” of 10 to 15% ethanol in 36 to 72 hours.

One assay used to assess total raw starch hydrolytic activity in whole culture enzyme preparations was a standardized simultaneous hydrolysis and fermentation. This assay is described in more details in the Examples. This assay was the most useful for assessing efficiency of raw starch activity as it is a measure of overall conversion of raw grain starch to ethanol. Other assays were used to evaluate enzyme preparations but no one assay predicted efficiency of raw starch conversion. Other assays used are described in the Examples and below.

The enzyme activities contained in the preparations were defined by selective substrate enzyme assays as described above and found to include alpha-amylases, glucoamylases, debranching (alpha 1,6 linkage) enzymes and beta glucanases. As noted herein, enzyme preparations that require no further purification find particular use in the invention. Hydrolysis of raw, granular starch is determined in assays measuring glucose and total soluble sugars from raw starch granules and by observing pitting and disappearance of raw starch in microscope examination. Optimal raw starch hydrolysis activity is at pH 3.5 to 3.8. Raw starch hydrolysis occurs at 10 to 50° C. It is not necessary to heat the starch to elevated temperatures to just below the gelatinization temperature of starch. The enzyme preparation hydrolyzes raw starch from any grain, grain waste material (for example residual material from manufacture of pearled barley) potatoes, or any other starch containing material. The beta glucanase content is a particular advantage with barley feedstock. These enzymes hydrolyze the beta glucans contained in barley, which create high mash viscosity in barley mash that increases capital and operating costs.

One component of the enzyme composition is alpha-amylase. By “α-amylase (e.g., E.C. class 3.2.1.1)” herein is meant enzymes that catalyze the hydrolysis of alpha-1,4-glucosidic linkages (thus also known as 1,4-α-D-glucan glucanohydrolase; glycogenase). These enzymes have also been described as those effecting the exo or endohydrolysis of 1,4-α-D-glucosidic linkages in polysaccharides containing 1,4-α-linked D-glucose units. By acting at random locations along the starch chain, α-amylase breaks down long-chain carbohydrates, ultimately yielding maltotriose and maltose from amylose, or maltose, glucose and “limit dextrin” from amylopectin. Because it can act anywhere on the substrate, α-amylase tends to be faster acting than β-amylase. Another term used to describe these enzymes is “glycogenase”. Exemplary enzymes include alpha-1,4-glucan 4-glucanohydrase glucanohydrolase.

The alpha-amylase of the invention is characterized by its ability to hydrolyze carbohydrates under acidic conditions. An amylase produced by fungi and able to hydrolyze carbohydrates under acidic conditions is referred to herein as acid fungal amylase, and is also known as an acid stable fungal alpha-amylase. Acid fungal amylase can catalyze the hydrolysis of partially hydrolyzed starch and large oligosaccharides to sugars such as glucose. The acid fungal amylase that can be employed in the present process can be characterized by its ability to aid the hydrolysis of raw or native starch, enhancing the saccharification provided by glucoamylase

Alpha amylase activity may be measured by using the DNS method as described in Miller, G. L. (1959) Anal. Chem. 31:426 428, U.S. Patent Application Publication No. 20030125534, and Food And Nutrition Board, National Research Council, Food Chemicals Codex (5th ed. 2003) (hereinafter Food Chemicals Codex) herein all incorporated by reference. The amount of acid fungal amylase employed in the present process can vary according to the enzymatic activity of the enzyme preparation. In general, activities of 40 to 70 alpha amylase units as defined by the assay method described in Example 4 were used. Other assays that can be used are the soluble substrate assay and starch hydrolysis assay described in U.S. Pat. No. 5,736,499, herein is incorporated by reference in its entirety. Additional assays are described in more detail in the Examples.

The enzyme composition provided in the present invention also optionally comprises “glucoamylase.” By “glucoamylase” herein is meant the amyloglucosidase class of enzymes (e.g., EC.3.2.1.3, glucoamylase, 1,4-alpha-O-glucan glucohydrolase), an enzyme that removes successive glucose units from the non-reducing ends of starch. These are exo-acting enzymes, which release glucosyl residues from the non-reducing ends of amylose and amylopectin molecules. The enzyme also hydrolyzes alpha-1,6 and alpha-1,3 linkages although at much slower rate than alpha-1,4 linkages. Glucoamylases are produced by several filamentous fungi and yeasts, with those from Aspergillus being commercially most important.

Glucoamylase activity may be assayed by the 3,5-dinitrosalicylic acid (DNS) method. Goto et al., Biosci. Biotechnol. Biochem. 58:49-54 (1994). The amount of glucoamylase employed in the present process can vary according to the enzymatic activity of the enzyme preparation. In general, activities of 300 to 500 glucoamylase units as defined by the assay method described in Example 4 were used. Additional assays are described in more detail in the Examples.

The enzyme composition provided herein may also include beta-glucanases. By “beta-glucanase” herein is meant the enzyme that can digest beta glucan, such as the beta glucan from barley.

Method for assaying beta-glucanase can also be found in Food Chemicals Codex and Walsh et al., Journal of Animal Science, 73(4):1074-1076 (1995), herein are incorporated by reference. Additional assays are described in more detail in the Examples.

Additional enzymes may be added in the compositions and methods encompassed by the invention.

In some embodiments, extra enzymes may be added to the enzyme preparations of the present invention. These enzymes include both carbohydrases as well as additional enzymes.

In some embodiments, for example, additional carbohydrases can be added, for example, additional α-amylase(s) can be added. Fungal amylase can be isolated from any of a variety of fungal species, including Aspergillus, Rhizopus, Mucor, Candida, Coriolus, Endothia, Enthomophtora, Irpex, Penicillium, Sclerotium and Torulopsis species. In an embodiment, the acid fungal amylase is thermally stable and is isolated from Aspergillus species, such as A. niger, A. saitoi or A. oryzae, from Mucor species such as M. pusillus or M. miehei, or from Endothia species such as E. parasitica. In an embodiment, the acid fungal amylase is isolated from Aspergillus niger. In addition, many of these fungal enzymes, including α-amylase, can be purchased and added to the processes of the invention; see for example, SPEZYME® series of thermostable alpha-amylase and DISTILLASE® series of glucoamylase from Genencor International Inc., and Spirizyme® brands of glucoamylase and Termamyl® brands of alpha-amylase by Novozymes Inc.

Another carbohydrase enzyme that may be added to the compositions of the invention are beta-amylases (E.C. 3.2.1.2). These are exo-acting maltogenic amylases, which catalyze the hydrolysis of 1,4-alpha-glucosidic linkages in amylose, amylopectin and related glucose polymers.

Additional carbohydrase enzymes include but are not limited to debranching enzymes such as pullulanases (E.C. 3.2.1.41) and isoamylases (E.C. 3.2.1.68). Such enzymes hydrolyze alpha-1,6-glucosidic bonds. Thus, during the hydrolysis of the starch, debranching enzymes remove successive glucose units from the non-reducing ends of the starch.

Further additional enzymes which may be used are proteases, such as fungal and bacterial proteases. Fungal proteases include for example, those obtained from Aspergillus, Mucor and Rhizopus, such as A. niger, A. awamori, A. oryzae and M. miehei. Other enzymes include but are not limited to cellulases, hemicellulases, lipases cutinases, and lignase.

The effective amount of these enzymes to be included in the methods of the invention can be readily determined by one skilled in the art.

In some embodiments, an antimicrobial may be added to the compositions and fermentation medium of the invention. Antimicrobials are compounds that kill or inhibit the growth of microorganisms.

### II. Ethanol Production Process

### A. Conventional Ethanol Production Process

Conventional industrial ethanol plants employ amylase (starch degrading) enzymes in a multi-step process. First, a slurry or “mash” is made and then heated to 105° C. to 120° C. to hydrate and gelatinize the starch granules. In grain (or any natural source of plant starch) starch is contained in insoluble granules. This cooking step is necessary to hydrate the starch granules to make the starch accessible to the enzymes.

By “mash” herein is meant to a mixture of a fermentable carbon source (carbohydrate) in water used to produce a fermented product, such as an alcohol. Specifically, it refers to a mixture of hot water and crushed grain, which is can also be used to produce malt beverages. In industrial ethanol production, mash typically contains 3 to 35% solids for corn and a maximum of about 25% solids for barley.

The mash is then cooled to a lower temperature, typically about 98° C. and alpha amylase enzyme is added to break the starch polymer into short chains of glucose. It is further cooled to 35° C. to 65° C. and then glucoamylase is added to produce individual glucose molecules. The temperature at which the enzymes are added is dependent on the heat tolerance of the enzymes used. It is cooled again to approximately 30° C. and yeast is added to convert the glucose to ethanol (fermentation). Fermentation of the sugars generates metabolic heat which is removed from the process. After fermentation, the fermentation mixture called beer typically contains approximately 12% ethanol. This mixture (beer) is distilled to concentrate the ethanol. The non-starch portion of the grain is carried through the entire process and is recovered after distillation to make distillers dry grains (or DDGs).

Conventional saccharification uses liquefaction of gelatinized starch to create soluble dextrinized substrate which glucoamylase enzyme hydrolyzes to glucose. This process is also called “cooking.”

For example, U.S. Patent Application Publication No. 20040219649 describes a process where the mash is held at elevated temperature but below the gelatinization temperature for a period of time, followed by cooling and addition of raw starch enzymes and yeast for simultaneous hydrolysis and fermentation.

In comparison, the present invention provides processes that do not require the mash to be held at an elevated temperature. The enzymes are also functionally different since the mash does not need to by gelantanized by high temperature in the process provided by the present invention to make it available the starch available to the enzymes. As such, equipment costs and energy costs are less with the ATSH process provided herein.

### B. Ambient Temperature Starch Hydrolysis

The present invention provides Ambient Temperature Starch Hydrolysis (“ATSH”) processes for ethanol production from plant material.

The starting plant material is generally processed to produce a mash that has starch in a form more accessible and thus more easily converted than the starting plant material. By “plant material” herein is meant all or part of any plant (e.g., cereal grain), typically a material including starch. Suitable plant material can be any starch containing material, includes grains such as maize (corn, e.g., whole ground corn, either standard corn or waxy corn), sorghum (milo), waxy or standard barley, wheat, rye, rice, triticale and millet; and starchy root crops, tubers, or roots such as potato, sweet potato and cassava.

The present method converts starch from plant material (e.g., fractionated plant material) to ethanol. The plant material (e.g., fractionated plant material) can be reduced by a variety of methods, e.g., by grinding, to make the starch available for saccharification and fermentation. Other methods of plant material reduction are available. For example, vegetable material, such as kernels of corn, can be ground with a ball mill, a roller mill, a hammer mill, or another mill known for grinding vegetable material, and/or other materials for the purposes of particle size reduction. Also can be used is emulsion technology, rotary pulsation, sonication, magnetostriction, ferromagnetic materials, or the like. These methods of plant material reduction can be employed for substrate pretreatment. Although not limiting to the present invention, it is believed that these methods can increase surface area of plant material (e.g., fractionated plant material) while raising the effectiveness of flowing of liquefied media (i.e. decreased viscosity). These methods can include electrical to mechanical, mechanical to electrical, pulse, and sound based vibrations at varying speeds. This can provide varying frequencies over a wide range of frequencies, which can be effective for pretreating the plant material (e.g., fractionated plant material) and/or reducing particle size.

Although not limiting to the present invention, it is believed that certain of these sonic methods create low pressure around a particle of plant material (e.g., fractionated plant material) and induce cavitation of the particle or disruption of the particle structure. The cavitated or disrupted particle can increase availability of plant material (e.g., starch) to an enzyme, for example, by increasing surface area. It is believed that such pretreatment can decrease quantity of enzyme rates in the present method for ethanol production.

In one embodiment, the present method includes vibrating plant material (e.g., fractionated plant material) and cavitating the fluid containing the plant material. This can result in disrupting the plant material and/or decreasing the size of the plant material (e.g., fractionated plant material). In certain embodiments, the present method includes treating plant material (e.g., fractionated plant material) with emulsion technology, with rotary pulsation, with magnetostriction, or with ferromagnetic materials. This can result in disrupting the plant material and/or decreasing the size of the plant material (e.g., fractionated plant material). In another embodiment, the present method includes sonicating the plant material (e.g., fractionated plant material). This can result in disrupting the plant material and/or decreasing the size of the plant material (e.g., fractionated plant material).

In one embodiment, the present method can include employing sound waves for reducing plant material (e.g., fractionated plant material). The sound waves can be ultrasound. The present method can include sonicating the plant material (e.g., fractionated plant material). The method can include sonicating the plant material at a frequency (e.g., measured in kHz), power (e.g., measured in watts), and for a time effective to reduce (or to assist in reducing) the particle size to sizes described hereinabove. For example, the method can include sonicating the plant material (e.g., fractionated plant material) at 20,000 Hz and up to about 3000 W for a sufficient time and at a suitable temperature. Such sonicating can be carried out with commercially available apparatus, such as high powered ultrasonics available from ETREMA (Ames, Iowa).

In one embodiment, the present method can include employing rotary pulsation for reducing plant material (e.g., fractionated plant material). The method can include rotary pulsating the plant material (e.g., fractionated plant material) at a frequency (e.g., measured in Hz), power (e.g., measured in watts), and for a time effective to reduce (or to assist in reducing) the particle size to sizes described hereinabove. Such rotary pulsating can be carried out with known apparatus, such as apparatus described in U.S. Pat. No. 6,648,500, herein is incorporated by reference.

In an embodiment, the present method can include employing pulse wave technology for reducing plant material (e.g., fractionated plant material). The method can include rotary pulsing the plant material at a frequency (e.g., measured in Hz), power (e.g., measured in watts), and for a time effective to reduce (or to assist in reducing) the particle size to sizes described hereinabove. Such pulsing can be carried out with known apparatus, such as apparatus described in U.S. Pat. No. 6,726,133, herein is incorporated by reference.

A fine grind exposes more surface area of the plant material, or vegetable material, and can facilitate saccharification and fermentation. In one embodiment, the vegetable material is ground so that a substantial portion, e.g., a majority, of the ground material passes a sieve with a 0.1-0.5 mm screen. In another embodiment, about 35% or more of the ground vegetable material can fit through a sieve with a 0.1-0.5 mm screen. In yet another embodiment, about 35 to about 70% of the ground vegetable material can fit through a sieve with a 0.1-0.5 mm screen. In an embodiment, about 50% or more of the ground vegetable material can fit through a sieve with a 0.1-0.5 mm screen. In an embodiment, about 90% of the ground vegetable material can fit through a sieve with a 0.1-0.5 mm screen. In an embodiment, all of the ground vegetable material can fit through a sieve with a 0.1-0.5 mm screen. In another embodiment, about 70% or more, of the ground vegetable material can fit through a sieve with a 0.1-0.5 mm screen. In an embodiment, the reduced plant material (e.g., fractionated plant material) can be mixed with liquid at about 20 to about 50 wt-% or about 25 to about 45 wt-% dry reduced plant material (e.g., fractionated plant material). Optimum solids concentration depends on the starch content of the grain or other feedstock and ethanol tolerance of the yeast. The optimum starch content of the mash generally is the amount necessary to produce ethanol to the tolerance of the yeast. With most commercial distillery yeast maximum ethanol concentration is no more than 15%. Therefore if a beginning mash starch concentration exceeds about 30%, the amount over 30% would not be converted to ethanol by the yeast. On a mole weight basis, one mole of glucose or 180 grams converts to two moles of ethanol with a total gram molecular weight of 92 grams (46 grams per mole ethanol) 92/180=0.511. On a weight basis 51% of glucose converts to ethanol at theoretical yield)

The vegetable material can also be fractionated into one or more components. Any starch containing component can be employed in the process. For example, a vegetable material such as a cereal grain or corn can be fractionated into components such as fiber (e.g., corn fiber), germ (e.g., corn germ), and a mixture of starch and protein (e.g., a mixture of corn starch and corn protein). One of a mixture of these components can be fermented in a process according to the present invention. Fractionation of corn or another plant material can be accomplished by any of a variety of methods or apparatus. For example, a system manufactured by Satake can be used to fractionate plant material such as corn.

In one embodiment, the germ and fiber components of the vegetable material can be fractionated and separated from the remaining portion of the vegetable material. In another embodiment, the remaining portion of the vegetable material (e.g., corn endosperm) can be further milled and reduced in particle size and then combined with the larger pieces of the fractioned germ and fiber components for fermenting.

In one embodiment, the vegetable material can be milled to access value added products (such as neutraceuticals, leutein, carotenoids, xanthrophils, pectin, cellulose, lignin, mannose, xylose, arabinose, galactose, galacturonic acid, GABA, corn oil, albumins, globulins, prolamins, gluetelins, zein and the like).

Fractionation can be accomplished by any of a variety of methods and apparatus, such as those disclosed in U.S. Patent Application Publication No. 2004/0043117, the disclosure of which is incorporated herein by reference. Suitable methods and apparatus for fractionation include a sieve, sieving, and elutriation. Suitable apparatus include a frictional mill such as a rice or grain polishing mill (e.g., those manufactured by Satake, Kett, or Rapsco).

The prepared plant material (e.g., fractionated plant material) can be referred to as being or including “raw starch”. The starting plant material is generally processed to produce a mash that has starch in a form more accessible and thus more easily converted than the starting plant material. By “starch” herein is meant any material comprised of the complex polysaccharide carbohydrates of plants, comprised of amylose and amylopectin with the formula (C₆H₁₀O₅)ₓ, wherein X can be any number. In particular, “starch” refers to any plant-based material including, but not limited to, grains, grasses, tubers and roots and more specifically wheat, barley corn, rye, rice, sorghum, brans, cassava, millet, potato, sweet potato, and tapioca.

The present invention provides processes for converting starch (usually from processed plant material as outlined herein) to sugars that can be fermented by a microorganism such as yeast. This conversion can be carried out by saccharifying the reduced plant material with any of a variety of known saccharifying enzyme compositions.

By “hydrolysis”, “saccharification” or “saccharifying” herein is meant the process of converting starch to smaller polysaccharides and eventually to monosaccharides, such as glucose, with enzymes, e.g., glucoamylase and amylase. Conventional saccharification uses liquefaction of gelatinized starch to create soluble dextrinized substrate which glucoamylase enzyme hydrolyzes to glucose. In the present method, saccharification refers to converting raw starch to glucose with enzymes, e.g., glucoamylase and amylase. According to the present method, the raw starch is not generally subjected to conventional liquefaction and gelatinization to create a conventional dextrinized substrate, although as outlined herein, the enzyme preparations of the invention also find use in convention processes

In one embodiment, saccharification is conducted at a pH of about 6.0 or less, for example, about 3.5 to about 5.0, for example, about 3.5 to about 4.0, and preferably about 3.5.

In the present method, the raw starch is not subjected to conventional liquefaction and gelatinization to create a conventional dextrinized substrate, i.e., “without cooking.”

By “without cooking” herein is meant a process for converting starch to ethanol without significant heat treatment for gelatinization and dextrinization of starch using alpha-amylase. Generally, for the process of the present invention, “without cooking” refers to maintaining a temperature below starch gelatinization temperatures, so that saccharification occurs directly from the raw native insoluble starch to soluble glucose while bypassing conventional starch gelatinization conditions. Starch gelatinization temperatures are typically in a range of 57° C. to 93° C. depending on the starch source and polymer type. In the method of the present invention, dextrinization of starch using conventional liquefaction techniques is not necessary for efficient fermentation of the carbohydrate in the grain.

Saccharifying can be conducted without cooking. For example, saccharifying can be conducted by mixing source of saccharifying enzyme composition (e.g., the enzyme preparation provided herein), yeast, and fermentation ingredients with ground grain (e.g. the “mash”) and process waters without cooking.

In one embodiment, saccharifying can include mixing the processed plant material with a liquid, which can form a slurry or suspension and adding the enzyme preparations of the present invention to the liquid. Alternatively, the addition of the enzyme preparation can precede or occur simultaneously with mixing, in any order.

In one embodiment, the reduced plant material (e.g., fractionated plant material) can be mixed with liquid. In one embodiment, the solids concentration is such that the mash starch concentration is 25 to 30% (w/w) to produce a fermented beer with 10 to 15% (w/w) ethanol. As used herein, wt-% of reduced plant material in a liquid refers to the percentage of dry substance reduced plant material or dry solids. In one embodiment, the method of the present invention can convert raw or native starch (e.g., in dry reduced plant material) to ethanol at a faster rate at higher dry solids levels compared to conventional saccharification with cooking. Although not limiting to the present invention, it is believed that the present method can be practiced at higher dry solids levels because, unlike the conventional process, it does not include gelatinization, which increases viscosity.

Suitable liquids include water and a mixture of water and process waters, such as stillage (backset), scrubber water, evaporator condensate or distillate, side stripper water from distillation, or other ethanol plant process waters, as well as nutrient solutions disclosed herein. In one embodiment, the liquid includes water, In another embodiment, the liquid includes water in a mixture with about 1 to about 70 vol-% stillage, about 15 to about 60 vol-% stillage, about 30 to about 50 vol-% stillage, or about 40 vol-% stillage. In addition, solutions suitable as nutrient sources outlined above can be added here as well.

In the conventional process employing gelatinization and liquefaction, stillage provides nutrients for efficient yeast fermentation, especially free amino nitrogen (FAN) required by yeast. The present invention can provide effective fermentation with reduced levels of stillage and even without added stillage. In an embodiment, the present method employs a preparation of plant material (e.g., fractionated plant material) that supplies sufficient quantity and quality of nitrogen for efficient fermentation under high gravity conditions (e.g., in the presence of high levels of reduced plant material). Thus, in an embodiment, no or only low levels of stillage can suffice. Generally, stillage recycle for FAN is necessary in wet mill corn processes where the ethanol feedstock is a highly purified starch with little or no associated protein. When using whole grain in dry mill processes FAN is much less of an issue as the grain protein content is sufficient to support yeast growth and metabolism. However stillage recycle to reduce waste water treatment volume can be important in dry mill ethanol plant designs. The ATSH fermentation process provided herein can take place with or without stillage recycle in dry mill whole grain processes.

However, the present method provides the flexibility to employ high levels of stillage if desired. The present method does not employ conventional liquefaction. Conventional liquefaction increases viscosity of the fermentation mixture and the resulting stillage. The present method produces lower viscosity stillage. Therefore, in an embodiment, increased levels of stillage can be employed in the present method without detrimental increases in viscosity of the fermentation mixture or resulting stillage.

Further, although not limiting to the present invention, it is believed that conventional saccharification and fermentation processes require added FAN due to undesirable “Maillard Reactions” which occur during high temperature gelatinization and liquefaction. The Maillard Reactions consume FAN during cooking. As a result, the conventional process requires adding stillage (or another source of FAN) to increase levels of FAN in fermentation. It is believed that the present process avoids temperature induced Maillard Reactions and provides increased levels of FAN in the reduced plant material, which are effectively utilized by the yeast in fermentation.

Saccharification can employ any of a variety of known enzyme sources (e.g., a microorganism) or compositions to produce fermentable sugars from the reduced plant material (e.g., fractionated plant material). In an embodiment, the saccharifying enzyme composition includes an amylase, such as an alpha amylase (e.g., an acid fungal amylase) or a glucoamylase, and preferably, the enzyme preparation provided herein.

In an embodiment, saccharification is conducted at a pH of about 6.0 or less, pH of about 3.0 to about 6.0, about 3.5 to about 6.0, about 4.0 to about 5.0, about 4.0 to about 4.5, about 4.5 to about 5.0, or about 4.5 to about 4.8. In an embodiment, saccharification is conducted at a pH of about 4.1 to about 4.6 or about 4.9 to about 5.3. Preferred pH is 3.5 to 4 for simultaneous hydrolysis and fermentation at ambient temperature. The initial pH of the saccharification mixture can be adjusted by addition of, for example, ammonia, sulfuric acid, phosphoric acid, process waters (e.g., stillage (backset), evaporator condensate (distillate), side stripper bottoms, and the like), and the like.

The enzyme preparation provided in the present invention can be used as a replacement for expensive purified commercially available in conventional multiple-step process such that described in U.S. Patent Publication Nos: 20040234649, 20050233030, 20050239181, 20070036882, and 2007003267, herein all incorporated by reference. These publications relate to a process for converting raw starch to at least 15% ethanol with cooking. They disclose the methods of grinding the grain to a fine composition to allow a higher slurry concentration, and cooling the fermentation mash to allow the beer to reach at least 15% ethanol. There are few types of yeast that tolerate over 15% of ethanol at 30° C. The reduction in fermentation temperature increases the yeast tolerance to ethanol, allowing the beer concentration to reach 15%. Most yeasts are more tolerant of ethanol concentrations at lower temperatures, but the rate of conversion of sugar to ethanol decreases. Most yeast has an optimum performance around 30° C. Therefore, lower operating temperature may slow the fermentation process down. In addition, cooling the fermentation mash below 30° C. requires more energy and more equipment. However, in some embodiments, the present invention can be carried out at lower temperature to produce higher percentage of alcohol.

The sugar from starch hydrolysis is subjected to fermentation to produce ethanol. After the saccharification step is completed, the fermentable sugars are added to yeast where fermentation begins. Thus, the steps of starch hydrolysis and fermentation can be carried out separately. Alternatively, the steps of starch hydrolysis and fermentation can be carried out simultaneously. It is also possible to have overlapping steps of hydrolysis and fermentation. For example, the fermentation step can be initiated after the hydrolysis step starts, but before the hydrolysis is completed. This simultaneous saccharification and fermentation allows for higher concentrations of starch to be fermented.

### C. Fermentation

The process provided by the present invention includes fermenting sugars from the saccharification reaction utilizing the enzyme compositions of the invention to ethanol. Fermenting can be effected by a microorganism, such as yeast. The fermentation mixture need not, and in an embodiment does not, include protease. However, the process waters may contain protease. If optionally included, the amount of protease can be less than that used in the conventional process. According to the present invention, fermenting is conducted on a starch composition that has not been cooked. In an embodiment, the present fermentation process produces potable alcohol. Potable alcohol has only acceptable, nontoxic levels of other alcohols, such as fusel oils. Fermenting can include contacting a mixture including sugars from the reduced plant material (e.g., fractionated plant material) with yeast under conditions suitable for growth of the yeast and production of ethanol. In an embodiment, fermenting employs the saccharification mixture.

In one embodiment, fermentation is conducted at a pH of about 6 or less, for example, about 3.5 to about 5, for example, about 3.5 to about 4.5, and preferably about 3.5. In another embodiment, the present invention employs a batch process filling the fermentor in one step adjusting pH to about 3.5, pH rises to about 4 over the course of the hydrolysis fermentation. In another embodiment, the present method can include varying the pH. For example, fermentation can include filling the fermentor at pH of about 3 to about 4.5 during the first half of fill and at a pH of about 4.5 to about 6 (e.g., about 4.5 to about 4.8) during the second half of the fermenter fill cycle. The initial pH of the fermentation mixture can be adjusted by addition of, for example, ammonia, sulfuric acid, phosphoric acid, process waters (e.g., stillage (backset), evaporator condensate (distillate), side stripper bottoms, and the like), and the like.

Although not limiting to the present invention, it is believed that known distillery yeast grow well over the pH range of 3 to 6, but are more tolerant of lower pH's down to 3.0 than most contaminant bacterial strains. Contaminating lactic and acetic acid bacteria grow best at pH of 5.0 and above. Thus, in the pH range of 3.0 to 4.5, it is believed that ethanol fermentation will predominate because yeast will grow better than contaminating bacteria.

In an embodiment, the present method can include varying the pH. It is believed that varying the pH can be conducted to reduce the likelihood of contamination early in fermentation and/or to increase yeast growth and fermentation during the latter stages of fermentation. For example, fermentation can include filling the fermentor at pH of about 3 to about 4.5 during the first half of fill. Fermentation can include increasing the slurry pH to pH of about 4.5 to about 6 during the second half of the fermentor fill cycle. Fermentation can include maintaining pH by adding fresh substrate slurry at the desired pH as described above. In an embodiment, during fermentation (after filling), pH is not adjusted. Rather, in this embodiment, the pH is determined by the pH of the components during filling.

In one embodiment, the pH is decreased to about 5 or below in the corn process waters. In another embodiment, the pH is about pH 4 (e.g. 4.1) at the start of fermentation fill and is increased to about pH 5 (e.g. 5.2) toward the end of fermentation fill. In yet another embodiment, the method includes stopping pH control of the mash slurry after the yeast culture becomes established during the initial process of filling the fermentor, and then allowing the pH to drift up in the corn process waters during the end stages of filling the fermentor.

In one embodiment, fermentation is conducted at a temperature of about 25 to about 40° C. or about 30 to about 35° C. In another embodiment, during fermentation the temperature is decreased from about 40° C. to about 30° C. or about 25° C., or from about 35° C. to about 30° C., during the first half of the fermentation, and the temperature is held at the lower temperature for the second half of the fermentation. In one embodiment, fermentation is conducted for about 25 (e.g., 24) to about to 150 hours, for example, for about 48 (e.g., 47) to about 72 hours. In an embodiment, the temperature can be decreased as ethanol is produced. For example, in an embodiment, during fermentation the temperature can be as high as about 40° C. and then reduced to about 25° C. This temperature reduction can be coordinated with increased ethanol titers (%) in the fermentor.

In one embodiment, fermentation is conducted for about to 25 (e.g., 24) to about to 150 hours, about 25 (e.g., 24) to about 96 hours, about 40 to about 96 hours, about 45 (e.g., 44) to about 96 hours, about 48 (e.g., 47) to about 96 hours. For example, fermentation can be conducted for about 30, about 40, about 50, about 60, or about 70 hours. For example, fermentation can be conducted for about 35, about 45, about 55, about 65, or about 75 hours. Generally, fermentation can be conducted over any time period necessary to produce the maximum ethanol tolerated by the yeast and within practical economic limits. In practice the ATSH process of simultaneous hydrolysis and fermentation of raw starch uses an enzyme dose sufficient for a rate of hydrolysis that converts greater than 90% of theoretical starch to ethanol yield in 24 to 150 hour, and preferably 48 to 60 hours.

Any of a variety of yeasts can be employed as the yeast starter in the present process. Suitable yeasts include any of a variety of commercially available yeasts, such as commercial strains of Saccharomyces cerevisiae, Suitable strains include “Fali” (Fleischmann's), Thermosac (Alltech), Ethanol Red (LeSafre), BioFerm AFT (North American Bioproducts), and the like. In an embodiment, the yeast is selected to provide rapid growth and fermentation rates in the presence of ambient temperature and medium ethanol levels.

In one embodiment of the present invention, the yeast employed is a recombinant yeast having any number of different characteristics as required or desired. For example, yeast having enhanced stress resistance or increased tolerance to ethanol can be used. In another embodiment, the recombinant yeast exhibits a modified regulation of the expression of programmed cell death, including senescence. The amount of yeast starter employed is selected to effectively produce a commercially significant quantity of ethanol in a suitable time, e.g., less than 75 hours.

Yeast can be added to the fermentation by any of a variety of methods known for adding yeast to fermentation processes. For example, yeast starter can be added as a dry batch, or by conditioning/propagating. In one embodiment, yeast starter is added as a single inoculation. In an embodiment, yeast is added to the fermentation during the fermentor fill at a rate of 5 to 100 pounds of active dry yeast (ADY) per 100,000 gallons of fermentation mash. In another embodiment, the yeast can be acclimated or conditioned by incubating about 5 to 50 pounds of ADY per 10,000 gallon volume of fermentor volume in a prefermentor or propagation tank. Incubation can be from 8 to 16 hours during the propagation stage, which is also aerated to encourage yeast growth. The prefermentor used to inoculate the main fermentor can be from 0.1 to 10% by volume capacity of the main fermentor, for example, from 1 to 2% by volume capacity relative to the main fermentor.

One embodiment of the present invention is the use of complementary and synergistic yeast (enhanced fermenting microorganism) for fermentation improvements. The present invention, because it avoids the high viscosity created during conventional liquefaction based processes, allows the use of improved, stress resistant, fast growing yeast capable of producing ethanol under extreme high gravity conditions. An embodiment of the present invention includes the use of an enhanced fermenting microorganism with technology referenced in U.S. Pat. Nos. 6,878,860, 6,867,237, 6,855,529, 6,849,782, 6,774,284, and 6,538,182, all are incorporated herein by reference in their entirety and particularly for the strains and conditions outlined therein

Another embodiment of the present invention includes the use of a recombinant yeast microorganism having enhanced stress resistance, and exhibiting a modified regulation of the expression of programmed cell death, including senescence. In one embodiment, the recombinant yeast includes a gene or gene fragment that inhibits the expression and/or activity of a polypeptide whose expression is induced by the onset of apoptosis, or that mediates senescence. In another embodiment, the polypeptide that is inhibited is eukaryotic initiation Factor-5A (elF-5A). In one embodiment, the inhibited polypeptide is apoptosis-induced deoxyhypusine synthase (DHS). In another embodiment, the recombinant yeast may include a combination of genes or gene fragments that inhibit the expression and/or activity of more than one polypeptide. In yet another embodiment, the inhibition of the polypeptide results in alteration of the level of senescence.

In one embodiment, the present method includes solids staging. Solids staging includes filling at a disproportionately higher level of solids during the initial phase of the fermentor fill cycle to increase initial fermentation rates. The solids concentration of the mash entering the fermentor can then be decreased as ethanol titers increase and/or as the fermentor fill cycle nears completion. In an embodiment, the solids concentration can be about 40% (e.g. 41%) during the first half of the fermentation fill. This can be decreased to about 25% after the fermentor is 50% full and continuing until the fermentor fill cycle is concluded. In the above example, such a strategy results in a full fermentor with solids at 33%.

### D. Simultaneous Starch Hydrolysis and Fermentation

The present process can include simultaneously converting reduced plant material (e.g., fractionated plant material) to sugars and fermenting those sugars with a microorganism such as yeast. Simultaneous saccharification and fermentation can be conducted using the reagents and conditions described above for saccharification and fermentation. Preferably, the enzyme preparation provided in the present invention is used in the process of simultaneous hydrolysis and fermentation of uncooked starch mash as described in more details below.

The present invention provides simultaneous raw starch hydrolysis and fermentation process uses mash solids content standard in the industry to produce 12 to 15% (w/w) ethanol with mash temperature that never exceeds 40° C. But ideally it is operated at a temperature as high as possible to avoid cooling costs.

In one embodiment of the ATSH ethanol production process provided herein raw starch, water, enzyme preparation, and yeast are combined in a single step in one fermentation vessel. The resulting mash is kept at near ambient temperature and low pH. The ATSH enzyme preparation converts the raw, non-hydrated starch to glucose while the yeast simultaneously converts it to ethanol. An acidic pH of 3.5 controls bacterial contamination.

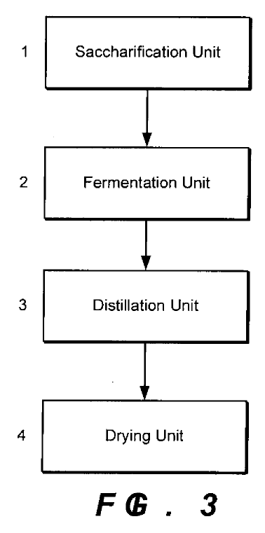

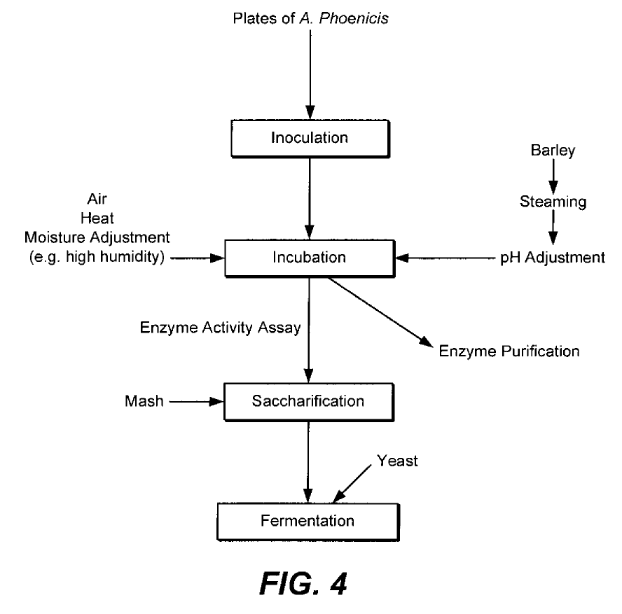

The diagram shown in FIG. 2 provides a comparison between the convention ethanol production process and the ATSH ethanol production process.

The ATSH process provided herein eliminates the cooking and cooling of the mash associated with a conventional ethanol process. This reduces capital cost through the elimination of jet cookers, heat exchangers, and reduces boiler size. The simultaneous hydrolysis and fermentation generates less heat than a straight fermentation process where all the glucose is made prior to the addition of yeast. The simultaneous hydrolysis and fermentation reduce fermentation cooling costs.

In a conventional ethanol plant, energy is used in five major areas: (1) cooking the mash; (2) cooling the mash; (3) cooling the fermentation; (4) distillation; and (5) drying the distillers grains.

Conventional ethanol plants do an excellent job or recovering waste heat. Waste heat from the distillation process is used to preheat the mash prior to the jet cooking step. Cold water is used to remove heat from the mash after cooking and during fermentation. Large cooling towers are used to remove heat from the cooling water. Steam is used to heat water for distillation and to dry the distillers grain.

However, the ATSH process eliminates the cooking and reduces cooling costs. This results in capital savings and operating cost savings. Waste heat from distillation can be used to preheat the beer prior to distillation and to cut drying expenses for DDG's.

With the typical known systems for producing ethanol from starch, e.g. using a dual enzyme system for liquefying and saccharifying the starch to glucose followed by batch fermentation, total processing times of 60 to 80 hours are usual. Fermentation times of 50 to 72 hours are commonplace. Such long total residence times result in enormous tankage requirements within the processing system when large scale ethanol production is contemplated.

In the fermentation process, yeast is added to a solution of simple sugars. Yeast is a small microorganism which uses the sugar in the solution as food, and in doing so, expels ethanol and carbon dioxide as byproducts. The carbon dioxide comes off as a gas, bubbling up through the liquid, and the ethanol stays in solution. Unfortunately, the yeast stagnates when the concentration of the ethanol in solution approaches about 12% to 18% (volume/volume), whether or not there are still fermentable sugars present.

In order for nearly complete fermentation, and in order to produce large quantities of ethanol, the common practice has been to use a batch process wherein extremely large fermentation vessels capable of holding upwards of 500,000 gallons are used. With such large vessels, it is economically unrealistic to provide an amount of yeast sufficient to rapidly ferment the sugar solution. Hence, conventional fermentation processes have required 72 hours and more because such time periods are required for the yeast population to build to the necessary concentration. For example, a quantity of yeast is added to the fermentation vessel. In approximately 45-60 minutes, the yeast population will have doubled; in another 45-60 minutes that new yeast population will have doubled. It takes many hours of such propagation to produce the quantity of yeast necessary to ferment such a large quantity of sugar solution.

The sugars used in traditional fermentation processes have typically contained from about 6 percent to 20 percent of the larger, complex sugars, such as dextrins and dextrose, which take a much longer time to undergo fermentation if they will undergo fermentation, than do the simple hexose sugars, such as glucose and fructose. Thus, it is common practice to terminate the fermentation process after a specified period, such as 72 hours, even though not all of the sugars have been utilized.

The present invention provides an ethanol production process—simultaneous hydrolysis and fermentation of uncooked mash. In this ethanol production process raw starch active enzyme and yeast are combined with the mash in a single step in one fermentation vessel with fermentation at ambient temperature and low pH.

One of the important concerns with conventional fermentation systems is the difficulty of maintaining a sterile condition free from bacteria in the large-sized batches and with the long fermentation period. Unfortunately, the optimum atmosphere for fermentation is also extremely conducive to bacterial growth. Should a batch become contaminated, not only must the yeast and sugar solution be discarded, but the entire fermentation vessel must be emptied, cleaned, and sterilized. Such an occurrence is both time-consuming and very costly.

Additionally, many of these bacteria compete with the yeast for sugar, thereby reducing the amount of ethanol that is produced. Bacteria can grow nearly ten times faster than yeast, thus contamination in these areas are inevitable. Upon the consumption of sugar, these bacteria produce lactic acid and other byproducts. Further, if the fermentation vessels are not properly disinfected or sterilized between batches or uses, bacteria and other undesirable microorganisms can become attached to the interior walls of the fermentation vats where they will grow and flourish. These undesirable microorganisms may contaminate ethanol co-products such as animal feed, or they may consume valuable quantities of the substrate, or sugar, thus reducing the production of ethanol. The economics and efficiency of fermentation processes are frequently such that they cannot tolerate any such loss of production.

During the manufacturing of fuel ethanol, as for the enzyme production, bacteria contamination may occur.

Current methods used to kill these unwanted microorganisms, among others, often involve introduction of foreign agents, such as antibiotics, heat, and strong chemical disinfectants, to the fermentation before or during production of ethanol.

In one aspect of the present invention, the temperature is shifted during the fermentation.

In one embodiment, saccharification and fermentation is conducted at a temperature of about 25 to about 40° C. or about 30 to about 35° C. In another embodiment, during saccharification and fermentation the temperature is decreased from about 40 to about 25° C., or from about 35 to about 30° C. during the first half of the saccharification, and the temperature is held at the lower temperature for the second half of the saccharification.

Without being bound by theory, it is believed that higher temperatures early during saccharification and fermentation can increase conversion of starch to fermentable sugar when ethanol concentrations are low. This can aid in increasing ethanol yield. At higher ethanol concentrations, this alcohol can adversely affect the yeast. Thus, it is believed that lower temperatures later during saccharification and fermentation are beneficial to decrease stress on the yeast. This can aid in increasing ethanol yield. Initial fermentation temperature could be up to 40° C. depending on the yeast strain used and reduced as ethanol increases to about 15 to 20° C. to achieve 15% or more ethanol concentration. We prefer to run the simultaneous hydrolysis and fermentation at about 35° C. to 12 to 14% ethanol. Cooling for high ethanol concentration increases fermentation time.

Without being bound by theory, it is believed that higher temperatures early during saccharification and fermentation can reduce viscosity during at least a portion of the fermentation. This can aid in temperature control. It is also believed that lower temperatures later during saccharification and fermentation are beneficial to reduce the formation of glucose after the yeast has stopped fermenting. Glucose formation late in fermentation can be detrimental to the color of the distillers dried grain co-product.

In another aspect of the present invention, the temperature is kept constant during fermentation, thus eliminate the costs of heating and cooling. It is also noted that due to the heat generated by the fermentation process it self, the temperature may shift nevertheless. Thus keeping the fermentation temperature constant should be understood as a relative term, refers to a process without actively shifting the temperature by external heating and cooling.

In one embodiment, simultaneous saccharification and fermentation is conducted at a pH of about 6 or less, pH of about 3 to about 6, about 3.5 to about 6, about 3.5 to 4.0. The initial pH of the saccharification and fermentation mixture can be adjusted by addition of, for example, ammonia, sulfuric acid, phosphoric acid, process waters (e.g., stillage (backset), evaporator condensate (distillate), side stripper bottoms, and the like), and the like. In one embodiment, saccharification and fermentation is conducted for about to 25 (e.g., 24) to about to 150 hours, about 25 (e.g., 24) to about 72 hours, about 45 to about 55 hours, about 50 (e.g., 48) to about 96 hours, about 50 to about 75 hours, or about 60 to about 70 hours. For example, saccharification and fermentation can be conducted for about 30, about 40, about 50, about 60, or about 70 hours. For example, saccharification and fermentation can be conducted for about 20 to 168 hours preferably about 48 to 72 hours. Generally, 48 to 72 hours to obtain 12 to 15% ethanol is economically competitive.

In one embodiment, simultaneous saccharifying and fermenting can be carried out employing quantities of enzyme preparation and yeast selected to maintain high concentrations of yeast and high levels of budding of the yeast in the fermentation broth. For example, the present process can employ quantities of enzyme preparation and yeast selected to maintain yeast at or above about 200 cells/mL, at or above about 300 cells/mL, at about 300 to about 600 cells/mL, or at about 100,000 to one million cells per ml.

In one embodiment, simultaneous saccharifying and fermenting can be carried out employing quantities of enzyme preparation and yeast selected for effective fermentation without added exogenous nitrogen; without added protease; and/or without added backset, and any and all combinations. Backset can be added, if desired, to consume process water and reduce the amount of wastewater produced by the process. In addition, the present process maintains low viscosity during saccharification and fermentation.

In one embodiment, simultaneous saccharifying and fermenting can be carried out employing quantities of enzyme preparation and yeast selected to maintain low concentrations of soluble sugar in the fermentation broth. In another embodiment, simultaneous saccharifying and fermenting can be carried out employing quantities of enzyme preparation and yeast selected to maintain low concentrations of glucose in the fermentation broth. For example, the present process can employ quantities of enzyme preparation and yeast selected to maintain glucose at levels at or below about 2 wt-%, at or below about 1 wt-%, at or below about 0.5 wt-%, or at or below about 0.1 wt-%. For example, the present process can employ quantities of enzyme and yeast selected to maintain glucose at levels at or below about 2 wt-% during saccharifying and fermenting. For example, the present process can employ quantities of enzyme and yeast selected to maintain glucose at levels at or below about 2 wt-% from hours 0-10 (or from 0 to about 15% of the time) of saccharifying and fermenting. For example, the present process can employ quantities of enzyme and yeast selected to maintain glucose at levels at or below about 1 wt-%, at or below about 0.5 wt-%, or at or below about 0.1 wt-% from hours 12-54 (or from about 15% to about 80% of the time) of saccharifying and fermenting. For example, the present process can employ quantities of enzyme and yeast selected to maintain glucose at levels at or below about 1 wt-% from hours 54-66 (or about from 80% to about 100% of the time) of saccharifying and fermenting. In the simultaneous hydrolysis and fermentation with high yeast loading, generally there is essentially no measurable glucose or maltose during the fermentation. With low yeast loadings or with delayed addition of yeast, glucose concentrations may be up to 4%.

The amount of enzyme of preparation can be adjusted as to generate optimal output. For example, simultaneous saccharifying and fermenting can employ enzyme preparation at about 0.1-10% w/w, preferably 0.25 to 5% 1-2% of dry solids reduced plant material. In general, enzyme dose is set to achieve 90% or greater of theoretical starch to ethanol conversion in 48 to 60 hours.

The saccharification and/or fermentation mixture can include additional ingredients to increase the effectiveness of the process. For example, the mixture can include added nutrients (e.g., yeast micronutrients), antibiotics, salts, added enzymes, and the like. Nutrients can be derived from stillage or backset added to the liquid or other sources. Suitable salts can include calcium, zinc or magnesium salts, such as calcium chloride, zinc sulfate, magnesium sulfate, and the like. Suitable added enzymes include those added to conventional processes, such as protease, phytase, cellulase, hemicellulase, exo- and endo-glucanase, xylanase, and the like.

In simultaneous hydrolysis and fermentation, the enzyme converts starch in starch granules to glucose, which the yeast immediately ferments to ethanol. In the process provided in the present invention the mash is generally adjusted to pH 3.5 to 4.0 with the addition of acid to inhibit contaminating bacteria. The present invention employs commercially available yeast in the process. Temperature is controlled generally at between 20 and 40° C., typically at about 35° C., optimal for most commercial distillery yeast. The concentration of starch in the mash and ratio of enzyme to starch determines the final concentration rate of ethanol production. Grain mash can be up to 40% solids, final ethanol concentration up to 14% v/v and total hydrolysis fermentation time from 26 to 72 hours. Overall starch to ethanol conversion efficiency generally exceeds 90% of theoretical and is generally equal to conversion efficiency of conventional cooking processes.

The inventors have run the ATSH process with corn, barley and wheat. Overall starch to ethanol conversion efficiency exceeds 90% of theoretical and is equal to conversion efficiency of conventional cooking processes. The ATSH process can produce 2.0 to 2.2 gallons of ethanol per bushel of barley, 2.7 gallons per bushel of corn, and 2.5 gallons per bushel of wheat.

The concentration of starch in the mash and ratio of enzyme to starch determines the final concentration rate of ethanol production. Grain mash can be up to 40% solids, final ethanol concentration up to 14% v/v and total hydrolysis fermentation time from about 24 to 72 hours. The time is a function of the enzyme and starch concentrations.

The product of the fermentation process is referred to herein as “beer”. For example, fermenting corn produces “corn beer”. Ethanol can be recovered from the fermentation mixture, from the beer, by any of a variety of known processes. For example, ethanol can be recovered by distillation.

The remaining stillage includes both liquid and solid material. The liquid and solid can be separated by, for example, centrifugation. The recovered liquid, thin stillage, can be employed as at least part of the liquid for forming the saccharification and fermentation mixture for subsequent batches or runs.

The recovered solids, distiller's dried grain, include unfermented grain solids and spent yeast solids. Thin stillage can be concentrated to a syrup, which can be added to the distiller's dried grain and the mixture then dried to form distiller's dried grain plus solubles. Distiller's dried grain and/or distiller's dried grain plus solubles can be sold as animal feed.

### E. Burn-Out of Residual Starches for Subsequent Secondary Fermentation

In one embodiment, the present method can include heat treatment of the beer or stillage, e.g., between the beer well and distillation. In another embodiment, the present method can include heat treatment of the beer or stillage and enzyme addition, e.g., between the beer well and distillation. This heat treatment can convert starches to dextrins and sugars for subsequent fermentation in a process known as burn-out. Such a treatment step can also reduce fouling of distillation trays and evaporator heat exchange surfaces. In yet another embodiment, heat treatment staging can be performed on whole stillage or thin stillage. Following enzymatic treatment of the residual starches, in an embodiment, the resulting dextrins and sugars can be fermented within the main fermentation process as recycled backset or processed in a separate fermentation train to produce ethanol. In one embodiment, the liquefaction and saccharification on whole stillage or thin stillage produced by centrifugation can be accelerated after distillation.

### F. Continuous Fermentation

The process provide herein can be run via a batch or continuous process. A continuous process includes moving (pumping) the saccharifying and/or fermenting mixtures through a series of vessels (e.g., tanks) to provide a sufficient duration for the process. For example, a multiple stage fermentation system can be employed for a continuous process with 48-96 hours residence time. For example, reduced plant material (e.g., fractionated plant material) can be fed into the top of a first vessel for saccharifying and fermenting. Partially incubated and fermented mixture can then be drawn out of the bottom of the first vessel and fed in to the top of a second vessel, and so on.

Although not limiting to the present invention, it is believed that the present method is more suitable than conventional methods for running as a continuous process. It is believed that the present process provides reduced opportunity for growth of contaminating organisms in a continuous process. At present, the majority of dry grind ethanol facilities employ batch fermentation technology. This is in part due to the difficulty of preventing losses due to contamination in these conventional processes. For efficient continuous fermentation using traditional liquefaction technology, the conventional belief is that a separate saccharification stage prior to fermentation is necessary to pre-saccharify the mash for fermentation. Such pre-saccharification insures that there is adequate fermentable glucose for the continuous fermentation process.

The present method achieves efficient production of high concentrations of ethanol without a liquefaction or saccharification stage prior to fermentation. This is surprising since this conventional wisdom teaches that it is necessary to have adequate levels of fermentable sugar available during the fermentation process when practiced in a continuous mode. In contrast the present method can provide low concentrations of glucose and efficient fermentation. In the present method, it appears that the glucose is consumed rapidly by the fermenting yeast cell. It is believed that such low glucose levels reduce stress on the yeast, such as stress caused by osmotic inhibition and bacterial contamination pressures.

### III. Strain Selection

In one aspect, the present invention provides a method for selecting additional strains to produce enzymes for production of ethanol from starch. This method is used to adapt fungus to produce enzyme preparation for optimal production of sugar from raw starch under low pH condition and ambient temperature.

To that end, the present invention provides a method for selecting/adapting fungus that can grow at low pH and produce enzymes that can hydrolyze raw starch effectively at low pH. This method does not have to involve any genetic engineering.

By “selection” or “adaptation” herein is meant the process to obtain a clone or a strain of fungus that can grow in low pH and preferably produce the desired enzymes that function effectively under low pH.

By “clone” herein is meant the cells that are derived from a single parent cell. Normally, there is no genotype and/or phenotype difference between the parent cell and its clones. On molecular level, a clone of a parent cell should have the identical genome as the parent cell. In the case of micro organisms such as fungi that produce haploid asexual spores, this process is remarkably simple and essentially only requires the inoculation of the appropriate medium.

By “strain” herein is meant a genetic variant or subtype of a fungus. Thus, there is genotype and/or phenotype difference between a strain and the parent strain from which it is derived. The creation of a new strain can due to either naturally occurred mutations or artificially introduced mutations.

Without being bound to the theory, it is possible that the same strain becomes adapted to grow under low pH and produce enzymes that can convert starch to sugar under low pH. Thus, there may not be genotype changes. Alternatively, there may be one or more mutations occurred at the gene and/or protein level that result in certain clones that are better fit to grow at lower pH. Thus a new strain is obtained.

It is possible to compare the parent strain and the strains that adapted to grow at low pH by methods known in the art, such as genome sequencing. However, this may not be necessary because such determination in some case can be costly and time consuming.

For ease of description, the present invention does not distinguish between parent strain and the progeny strain which may be derived through the selection process described herein also as a strain (a progeny strain). Thus it is possible that the progeny strain is only a clone of the parent strain and is not a new strain under the strict definition of microbiology.

The selection process starts with choosing a parent strain. The parent strain can be any strain that produce starch enzymes and which can be cultured from haploid condiospores. The selection/adaptation process provided in the instant invention can also be applied to select other strains of fungus to produce enzymes that can be used in the present invention Suitable fungal species include: Aspergillus quadricinctus, A. niger, A. oryzae, A. phoenicis, A. terreus, Rhizopus arrhizus, R. delemar, R. kasanensis, R. javonicus, R. oligosporus, R. oryzae and R. thailandensis.

In one embodiment, the strain selected as the best for the raw starch hydrolysis, Aspergillus phoenicis, ATCC 15556 was selected from screening studies of 26 strains of Aspergillus and Rhizopus judged from research literature, available toxicology information and preliminary screens as the best genera for selecting a fungal strain for producing raw starch active amylases with low pH optima.

In one embodiment, the parent strain is a strain of Aspergillus phoenicis (ATCC 15556) obtained from the American Type Culture Collection, 10801 University Blvd., Manassas, Va. 20110.

In one aspect of the present invention, the parent strain is first inoculated in a culture medium having a first pH value. The culture medium can be any medium known in the art that is suitable for the growth of the parent strain. For example, Aspergillus phoenicis can be grown in liquid culture in Czapek-Dox medium or on Czapek-Dox agar plates at 30° C. Alternatively, it can be grown on malt extract agar. These media are commercially available, such as from Merck KGaA, Darmstadt, Germany. In some embodiments, the fungus was grown on barley agar at 35° C. Barley agar consists of 5% finely ground barley in water with 15 to 30 grams per liter of agar with pH adjusted by addition of any acid most commonly sulfuric acid and autoclaved. Alternatively the selection substrate consisted of finely ground barley heat sterilized prior addition of water. In this procedure, starch is not hydrated and remains in intact “raw starch” granules. Sterile water containing variable concentrations of acid added to the dry starch to about 50% moisture final moisture.

The first pH could be the normal pH for growing the fungus. For example, generally Aspergillus phoenicis is grown on a Czapek-Dox medium of pH 7.3 or on barley agar. The first pH could be 7.3 or lower, such as 6. Thus one or more colonies from the parent strain (such as from an ATCC stock) can be used to inoculate a first plate with a first pH value. After the fungus has adapted to grow under the first pH value, one or more colonies from the first plate can be picked and used to inoculate a fresh second plate with a second pH value. The second pH value is normally lower than the first pH value, such as lower by 0.1 to 2.0, or by 0.5 to 1.0, and preferably by 0.5. Thus, if the first pH is 6, the second pH is preferably 5.5. After the fungus is adapted to grow under the second pH value, one or more colonies can be picked to inoculate a third plate with a third pH value. The third pH value is also generally lower than the second pH value, such as lower by 0.1 to 2.0, or by 0.5 to 1.0, and preferably by 0.5. Thus, if the second pH is 5.5, the third pH is preferably 5.0.

The above process can be repeated several times until the fungus is adapted to grow under a final pH value. By “final pH” or “final pH value” herein is meant the pH at which the fungus can grow and produce enzymes to convert starch to sugars at a pH low enough to prevent or reduce microbial contamination, e.g. low pH optima. By “low pH optima” herein is meant the pH under which an enzyme can function effectively to convert the substrate into final product, such as converting starch into sugar. The final pH value and the low pH optima of the enzyme do not have to be same. However, preferably, the final pH value is the same as, or is close to, the low pH optima. In one embodiment, both the final pH value and the low pH optima are about 3.5.

The process for identifying a likely strain involves compiling a data matrix for selecting and rapidly screening hundreds of strains to select a strain suitable for commercial use in ethanol production (or other application). Strain for screening are first selected from culture collections or isolated from natural habitats based on: 1) known or observed use of starch as a carbon source;) species that are not reported to have any adverse toxicity or pathogenicity, preferably species generally recognized as safe or with a history of industrial or food use; 3) species that produce stable haploid condiospores; and 4) species that grow and produce enzymes active under temperatures conducive to ethanol fermentation, typically 25 to 40° C. Strains are then screened on agar containing starch as the principal carbon source. Barley agar is suitable as a media for screening strains. Strains are selected based on growth rate under desired conditions of temperature and beginning pH. In one embodiment, a subset of strains is selected for adaptation on increasingly acidic agar as described herein. In another embodiment, a further subset of strains exhibiting rapid growth and adaptation to low pH is selected for evaluation in a standardized fermentation of raw starch. In one embodiment of the present invention this procedure was used in selecting a strain of A. phoenicis ATCC 15556 as the best of the strains producing raw starch active, low pH amylases

It is noted that the strain after the selection may or may not have genetic differences from the parent strain, or may not even have any other phenotype change aside from the ability to grow at low pH. In certain condition, the selected strain may be the same strain as the parent strain, only is adapted to be more tolerant of low pH growing conditions.

In one embodiment, a strain of Aspergillus phoenicis obtained from a public collection (ATCC 15556) was adapted for growth on starch at low pH. When grown in the SSC process as provided herein this strain produces a mixture of enzymes that hydrolyze the starch in raw starch granules at an optimum pH of 3.5.

Aspergillus is a genus of around 200 molds found throughout much of nature worldwide. Aspergillus species are highly aerobic and are found in almost all oxygen-rich environments, where they commonly grow as molds on the surface of a substrate, as a result of the high oxygen tension. Commonly, fungi grow on carbon-rich substrates such as monosaccharides (such as glucose) and polysaccharides (such as amylose). Aspergillus species are common contaminants of starchy foods (such as bread and potatoes), and grow in or on many plants and trees.

Aspergillus is used to make sake. First, koji mold such as Aspergillus oryzae is used to convert the starch in the rice to sugars (saccharification), which are subsequently fermented by other microorganisms, such as yeast Saccharomyces and lactic acid bacteria.

Aspergillus niger is the major source of citric acid; this organism accounts for over 99% of global citric acid production. Aspergillus niger is also commonly used for the production of native and foreign enzymes, including glucose oxidase and hen egg white lysozyme. In these instances, the culture is rarely grown on a solid substrate, but is more often grown as a submerged culture in a bioreactor. Aspergillus species grown in submerged culture are also used to produce amylases used commercially in converting starch to sugars.

A detailed description of the process for selecting the Aspergillus strain is provided in Example 1. One of the strains that obtained through this process is deposited in USDA ARS patent culture collection, strain number NRRL 50090.

### IV. System for Producing Ethanol

In an embodiment, the invention relates to a system that produces ethanol. A diagram of the system in shown in FIG. 3. The present system can include a saccharification unit 1, a fermentation unit 2, a distillation unit 3, and a dryer unit 4.

The saccharification unit 1 can be any of a variety of apparatus suitable for containing or conducting saccharification. The saccharification unit 1 can be, for example, a vessel in which reduced plant material can be converted to a sugar, which can be fermented by a microorganism such as yeast. The saccharification unit 1 can be configured to maintain a saccharification mixture under conditions suitable for saccharification. The saccharification unit 1 can be configured to provide for the conversion of reduced plant material with the addition of enzymes. In one embodiment, the saccharification unit 1 is configured for mixing reduced plant material with a liquid and adding a saccharifying enzyme composition to the liquid. In another embodiment, the saccharification unit 1 is configured for saccharification at a variety of pHs and temperatures, but preferably at a pH of 6.0 or less, and at a temperature of about 20 to about 50° C.

The fermentation unit 2 can be any of a variety of apparatus suitable for containing or conducting fermentation. The fermentation unit 1 can be, for example, a vessel in which sugar from reduced plant material can be fermented to ethanol. The fermentation unit 2 can be configured to maintain a fermentation mixture under conditions suitable for fermentation. In one embodiment, the fermentation unit 2 can be configured for fermenting through use of a microorganism, such as yeast or ethanol producing bacteria. In another embodiment, the fermentation unit 2 can be configured to ferment a starch composition that has not been cooked, specifically the saccharification mixture. In yet another embodiment, the apparatus can employ any variety of yeasts that yields a commercially significant quantity of ethanol in a suitable time Yeast can be added to the apparatus by any of a variety of methods known for adding yeast to a system that conducts fermentation. The fermentation unit 2 can be configured for fermentation for about 20 to 150 hours at a temperature of about 20 to about 40° C.

The saccharification unit 1 and the fermentation unit 2 can be a single, integrated apparatus. In one embodiment, this apparatus is configured to provide higher temperatures early on during simultaneous conversion of reduced plant material to sugars and fermentation of those sugars. In an embodiment, this apparatus is configured to provide lower temperatures later during the simultaneous saccharification and fermentation. The apparatus also may utilize the reagents and conditions described above for saccharification and fermentation, including enzymes and yeast.

The distillation unit 3 can be any of a variety of apparatus suitable for distilling products of fermentation. The distillation unit 3 can be, for example, configured to recover ethanol from the fermentation mixture (“beer”). In one embodiment, the fermentation mixture is treated with heat prior to entering the distillation unit 3. In another embodiment, fractions of large pieces of germ and fiber are removed with a surface skimmer or screen prior to or after entering the distillation unit 3.

The dryer unit 4 can be any of a variety of apparatus suitable for drying solids remaining after distillation (and optional centrifugation, for example, in a centrifuge system). In an embodiment, the dryer unit 4 is configured to dry recovered solids, which can result in production of distiller's dried grain. After the distillation system separates the ethanol from the beer, recovered solids remain. These recovered solids can then be dried in the dryer unit 4. This produces distiller's dried grain and/or distiller's dried grain plus solubles. In one embodiment, the dryer unit 4 can be or include a ring dryer. In another embodiment, the dryer unit 4 can be or include a flash dryer. In yet another embodiment, the dryer unit 4 can be or include a fluid bed dryer.

The examples provided herein are for illustration purposes only and are in no methods to limit the scope the present invention. Further, all references cited herein are incorporated by reference for all the relevant contents therein.

### EXAMPLES

### Example 1

### Selection of Aspergillus Strain

Many different species and strains of fungi produce extra cellular enzymes that hydrolyze starch. To develop enzyme preparations with specific and commercially useful characteristics, it is necessary to have a method to rapidly screen, adapt, and select specific strains. 26 strains of Aspergillus and Rhizopus were selected as the best genera for selecting a fungal strain for producing raw starch active amylases with low pH optima. Criteria for selecting strains for screening and adaptation were based on research literature, absence of reported adverse toxicology in literature review, reported use in production of enzymes or other uses approved for human consumption, production of haploid conidiospores, and preliminary screens of growth rate on starch media

Strains Strains included the following species: Aspergillus quadricinctus, A. niger, A. oryzae, A. phoenicis, A. terreus, Rhizopus arrhizus, R. dolemar, R., kasanensis. R. javonicus, R. otigosporus, R. oryzae and R. thailandensis. The strain selected as the best for the ATSH raw starch hydrolysis enzymes and ethanol process was Aspergillus phoenicis. One of the adapted strains has been deposited at USDA ARS patent culture collection as strain number NRRL 50090.

Materials culture media The starting cultures of Aspergillus phoenicis and other fungis were maintained on sterilized potato dextrose agar medium (Diced potato 200 g/l, Dextrose 20 g/l, and Agar 15 g/l), pH 4.5 and stored at 5° C. in the refrigerator. Conditioned strains were maintained on acid barley agar, pH 3.8. All the culture media, unless other wise stated, were sterilized in autoclave at 15-lbs/inch² pressure (121° C.) for 15 min. Cultures were maintained with periodic transfer to fresh media.

Barley agar consists of 50 grams per liter barley flour in water with 15 to 30 grams per liter agar was adjusted to pH 3.8 with sulfuric acid and sterilized by autoclaving at 15 psi 121° C.

Raw starch media consists of barley grain, ground to a fine powder. The dry ground barley was heat sterilized by autoclaving in a closed container or by heating to 120° C. in an oven. The ground barley was then mixed with sterile water or sterile water acid solution to 50% final moisture content. Preparation of this media did not hydrate and gelatinize starch granules.

Solid substrate culture media consists of barley flakes (hulled and steam rolled barley obtained from commercial sources) or extruded barley pellets, made by extruding a barley flour, water solution dough through an extruder so as to form a pellet about 2 to 5 mm in diameter and 5 to 10 mm in length. Barley flakes or pellets were adjusted to about 50% moisture content with a water solution containing: urea 16 grams per liter, Ammonium Phosphate 13.3 grams per liter, and sulfuric acid 13.3 ml one molar solution per liter of water. The pH of the solid substrate culture media is about 4.0. For laboratory scale SSC, 50 grams equivalent dry substrate or approximately 100 grams of moist substrate was place in beakers, autoclaved at 15 psi, 121° C. for 20 minutes, cooled, and inoculated with a selected fungal culture. Inoculated substrate was transferred under aseptic conditions to a SSC culture tubes (culture tubes are 1.25 inches OD by 10 inches long and about 200 cc working volume, however different size tubes can also be used). Tubes are capped at each end and fitted for air flow through the tube. Tubes were incubated at 30 to 35° C. in a culture rack with each tube having an independently controlled flow of humidified air. Air flow is about 10 to 20 cc/minute through the culture tubes.

After an incubation period of 3 to 10 days, cultures are removed from tubes, dried under a flow of dry air at 20 to 40° C., ground to a powder and stored at 4° C. until used for assay.

The basic assay procedure for efficient raw starch hydrolysis is a standardized, simultaneous hydrolysis fermentation of either barley or corn. Barley or corn was ground to pass a 20 mesh US standard screen. Standard media for hydrolysis fermentation is:

(1) Barley: 25 grams ground whole barley, 2.0 ml of a freshly grown yeast culture, 1.5 ml one molar sulfuric acid to adjust pH to about 3.6, and 0.1 to 1.0 grams of dry weight equivalent enzyme preparation (typically 0.5 grams or a 2% enzyme dose) and water to 100 grams total weight of fermentation.

(2) Corn: Alternatively 25 grams ground corn was used as the assay substrate with the same assay mix except that the acid volume was reduced to about 1.0 ml one molar sulfuric acid to adjust pH to 3.6.

Assays were conducted in 250 ml shake flasks incubated on a rotary shaker at 35° C. Fermentations were sampled at 24, 48 and 60 hours and assayed for ethanol concentration by gas chromatography. Whole culture enzyme preparations that produced greater than 6.5% ethanol in barley assays or 7.0% ethanol in corn assays at 60 hours with a 2% enzyme to substrate loading (0.5 grams whole culture enzyme preparation per 25 grams of dry weight grain) were judged to have sufficient raw starch activity. These ethanol concentrations represent greater than 90% of theoretical starch to ethanol conversion efficiency based on starch content of the barley or corn used in the assay. The rate of raw starch hydrolysis could be further evaluated by comparing ethanol concentration at 24 and 48 hours.

Selection of Fungal Strains Producing Raw Starch Active Amylases with pH Optima Less than 4.0.

To begin screening and adaptation to produce raw starch hydrolytic enzymes with pH optima less than 4 all strains were transferred to Petri plates of barley agar, adjusted to pH 6. After about three days of growth, a well sporulated section of the fungal culture was scrapped with an inoculation loop and transferred to barley agar at pH 5.5. Serial transfers to barley agar plates of decreasing pH was repeated in 0.5 pH units; pH 5.0. pH 4.5 to pH 4.0. Strains were sub-cultured twice on pH 4.0 agar then transferred to barley agar at pH 3.6, and sub cultured twice at this pH. Cultures were then transferred to barley agar slants at pH 3.6 to 3.8 for storage at 4° C. A subset of strains were also adapted in serial transfers of decreasing pH on the raw starch media to pH3.5 and then stored on barley agar pH 3.6 to 3.8 at 4° C.

To compare adapted cultures for raw starch hydrolysis, the acid adapted cultures were transferred from storage agar slants to fresh barley agar pH 3.6 and grown to uniform spore formation. Spores were then harvested from plates, suspended in 0.1% Tween 80 solution and used to inoculate SSC medium to about 1E6 spores per gram of substrate. SSC of different adapted and or unadapted strains were incubated as described above, harvested dried ground and compared in standard hydrolysis and fermentation. From this procedure several strains exhibited effective raw starch hydrolysis.

During the development of the low pH optima raw starch amylase 26 strains of Aspergillus and Rhizopus species were conditioned. Table 1 shows results of one experiment in which eight strains Aspergillus were grown in one culture set according to the methods described in Example 2 and evaluated in the same set of selective substrate enzyme assays and standardized hydrolysis and fermentation at pH 3.6 to 3.9 according to the methods of Examples 3 and 4. All strains had been conditioned to acidic conditions described above.

TABLE 1

| | | | | | Barley EtOH | | Corn EtOH | |
|---|---|---|---|---|---|---|---|---|
| Strain | AA | AM | MA | DeB | 16 | 64 | 16 | 64 |
| A1 | 34 | 30 | 164 | 240 | 2.4 | 5.3 | 1.9 | 5.9 |
| A3 | 52 | 75 | 181 | 218 | 2.6 | 6.4 | 2.7 | 6.7 |
| A5 | 42 | 60 | 85 | 184 | 0.1 | 5.9 | 2.0 | 6.3 |
| A6 | 71 | 56 | 54 | 102 | 0.6 | 0.9 | 0.8 | 1.0 |
| A7 | 69 | 55 | 54 | 99 | 0.6 | 1.0 | 0.9 | 1.3 |
| A8 | 62 | 82 | 271 | 268 | 2.6 | 6.5 | 2.7 | 7.0 |
| A16 | 59 | 78 | 198 | 196 | 2.6 | 5.9 | 2.4 | 6.3 |
| A18 | 43 | 41 | 110 | 162 | 2.2 | 5.0 | 1.8 | 5.3 |

Assays: AP Amylopectin Azure, AM Amylose Azure, MA Maltase, DeB Debranching, Barley EtOH standardized raw barley hydrolysis and fermentation ethanol concentration mg/ml at 16 an 64 hours, Corn EtOH standardized raw corn hydrolysis and fermentation ethanol concentration mg/ml at 16 an 64 hours.

Parent strains prior to acid conditioning:

A1 Aspergillus species

A2 Aspergillus niger

A3 Aspergillus niger NRRL 330

A5 Aspergillus niger NRRL 3536

A6 Aspergillus species

A7 Aspergillus niger

A8 Aspergillus awamori

A16 Aspergillus phoenicis NRRL QM 329

A18 Aspergillus quadricinctus ATCC 16897

The method of strain selection and acid adaptation allowed selection of a number of fungal strains that efficiently hydrolyzed raw granular starch. The strain finally selected as the most consistent in rapid ethanol production was acid conditioned culture of A. phoenicis ATCC 15556. This strain was not included in this comparison experiment. As shown in Table 2 below, this adapted strain produced 6.9 to 7.1 mg/ml ethanol in the standardized barley fermentation assay and 7.4 to 7.6 mg/ml ethanol in the standardized corn fermentation assay.

Example pH optima of adapted strain The adapted strain of A. phoenicis ATCC 15556 which showed the best results in the previous example was grown in SSC on barley flakes for 67 hours when cultures were harvested, dried and ground then evaluated for acid raw starch activity in standardized hydrolysis and fermentation of barley and corn at different pH. Results are shown below.

TABLE 2

| | | | wt % Ethanol Concentration | | | | |
|---|---|---|---|---|---|---|---|
| Fermen- | Initial | Final | at Hours Fermentation | | | | |
| tation | pH | pH | 16 | 24 | 40 | 48 | 64 |
| Barley | 3.3 | 3.9 | 2.6 | 3.8 | 5.1 | 5.9 | 7.1 |
| | 3.5 | 4.0 | 2.6 | 3.6 | 5.7 | 6.2 | 7.2 |
| | 3.7 | 4.1 | 2.5 | 3.7 | 5.4 | 6.0 | 6.9 |
| Corn | 3.3 | 3.9 | 3.0 | 4.4 | 6.0 | 6.7 | 7.5 |
| | 3.5 | 4.0 | 3.1 | 4.4 | 6.3 | 6.6 | 7.4 |
| | 3.7 | 4.1 | 2.7 | 4.4 | 6.3 | 6.4 | 7.6 |

### Example 2

### Laboratory Scale Solid Substrate Culture Production of Raw Starch Amylase

This example describes one embodiment for producing and analyzing an amylase preparation that hydrolyzes ungelatinized raw starch granules at near ambient temperature and composition of the enzyme preparations with multiple enzyme activities.

Inoculum was prepared from a stock spore culture of A. phoenicis. The spore preparation was prepared by using a pH 3.8 barley agar slant culture of A. phoenicis condition according to the methods of Example 1 to inoculate a laboratory solid substrate culture. The laboratory culture was made up of 50 grams dehulled steam rolled barley flakes wetted with 45 ml of nutrient solution consisting of:

| | Grams/liter |
|---|---|
| NH₄H₂PO₄ | 13 |
| Urea | 17 |
| H₂SO₄, 1 molar | 13 |

Wetted barley flakes were autoclaved at 121° C., 15 psi, cooled and inoculated from a barley agar slant. Inoculated substrate was then transferred to a 200 cc culture tube and incubated at 35° C. with a constant flow of humidified air for 4 days until heavily sporulated. The culture was then removed from the tube, dried at 40° C. in air and stored at 4° C. To inoculate solid culture, a ratio of 1 gram spore culture to 100 ml 0.1% Tween 80 in water was used. This produces a spore suspension of about 1×10⁸ spores per ml.

Solid Culture Substrate Preparation Substrate was dehulled steam rolled barley flakes, in this experiment for each culture 160 grams of as is weight flakes (about 5% moisture as received) were wetted with variable amounts of the nutrient solution in the table above. The flakes were soaked for one hour then steamed for 20 minutes at 95° C., cooled and inoculated with 10 ml of inoculum per 160 grams dry flakes. This experiment compared enzyme composition from cultures with three different rates of added moisture. Initial moistures including steaming and inoculation were 44%, 48% and 52%.

Culture incubation Inoculated substrate was transferred to 500 cc culture tubes. Tubes were 3.8 cm diameter by 45 cm long with caps at each end with fittings for introducing air flow at one end and exhausting air at the other. Tubes were incubated in a temperature controlled rack at 35° C. with a humidified air flow of 80 cc/minute per culture tube for 96 hours. Cultures were then removed and dried with a flow of dry air at 40° C. to a final moisture content of about 5%. This whole dried culture was then analyzed for multiple enzyme activities and for hydrolysis of barley in simultaneous hydrolysis and fermentation.

Composition The dry whole culture material recovered from the solid substrate culture constitutes the enzyme preparation. The enzyme preparation contains 30 to 60% of the original culture substrate on a dry weight basis; in this example total solid recovery was about 50%. The enzyme preparation contains residual starch, protein, fiber, and ash from the barley flakes, residual compounds from the added nutrients, the cell mass of the fungus, and enzyme protein.

### Example 3

### A Standardized Simultaneous Hydrolysis and Fermentation Assay

This assay used ground corn or barley and determined the ratio of enzyme to gain necessary to achieve at least 90% of theoretical starch to ethanol yield in 60 hours in simultaneous hydrolysis and fermentation conducted at 35° C. Standard hydrolysis and fermentation assays contained 25 grams ground whole barley, 2.0 ml of a freshly grown yeast culture, 1.5 ml one molar sulfuric acid to adjust pH to about 3.6, and 0.1 to 1.0 grams of dry weight equivalent enzyme preparation (typically 0.5 grams or a 2% enzyme dose) and water to 100 grams total weight of fermentation. Assays were conducted in 250 ml shake flasks incubated on a rotary shaker at 35° C. Fermentations were sampled at 24, 48 and 60 hours and assayed for ethanol concentration by gas chromatography. Alternatively, ground corn was used as the assay substrate with the same assay mix except that the acid volume was reduced to about 1.0 ml one molar sulfuric acid to adjust pH to 3.6. Whole culture enzyme preparations that produced greater than 6.5% ethanol in barley assays or 7.0% ethanol in corn assays at 60 hours were judged to have sufficient raw starch activity. Activity could be further evaluated by comparing ethanol concentration at 24 and 48 hours.

### Example 4

### Assays Used to Evaluate Enzyme Preparations

The following assays were used to evaluate enzyme preparations.

(1) Amylose azure/amylopectin azure The substrate is azure blue dye bound to either amylose (straight chain) or amylopectin (branch chain) starch. Azure blue starch is suspended in buffer at the desired pH in our case pH with enzyme added at the desired amount. The assay is incubated, stopped with addition of sodium carbonate, centrifuged to remove any solids and read on a spectrophotometer. Enzyme activity is proportional to the optical density from hydrolysis of the starch and release of blue dye. Units were arbitrarily defined as a change of 0.10 OD in a 15 minute assay conducted with 1% azure starch, 0.01 gram per ml enzyme preparation at pH 4 for 15 minutes. This assay measures predominantly alpha amylase activity from producing soluble short chain dextrins with attached blue dye.

(2) Polycose/maltose Polycose is a short chain soluble dextrin, average chain length is 5 glucose residues, maltose is two glucose residues. This assay employs either maltose or polycose dissolved in buffer at pH 3.8, enzyme added at 0.01 gram per ml of reaction mix and incubated at 35° C. Glucose was measured at 15 minutes using a glucose analyzer (Yellow Springs Instruments). This is an enzymatic assay specific for glucose and the assay measures primarily glucoamylase activity with release of glucose from short the short chain polycose or by splitting maltose. One unit is defined as release of production of 1 mg glucose per ml of reaction mixture in the 15 minute assay.

(3) Debranching activity This assay uses a highly branched starch containing a high proportion of alpha 1,6 linked glucose defining branch points in alpha 1,4 linked glucose polymers as substrate. The assay measured glucose as above.

(4) Beta glucanase activity This assay employed barley beta glucan purified from barley dissolved in buffer at pH 3.8, enzyme added at 0.01 gram per ml of reaction mix and incubated at 35 CC. Hydrolysis of beta glucan was assessed by the DNS method of the reaction mixture. DNS assay measures concentration of total soluble reducing sugars, in this case the total of glucose and soluble short chain glucose polymers produced by hydrolysis of beta glucan. Units are defined by optical density increase of 0.10 in a 15 minute assay; conducted at pH 4 in buffer, 35° C.

(5) Raw starch activity This assay used raw corn starch as substrate measuring total reducing sugar by DNS assay. Corn starch was incubated with 10% enzyme at pH 3.6, 35° C. for 30 minutes, centrifuged to remove solids and assayed for total soluble reducing sugars by DNS.

TABLE 3

| Culture/ | Alpha | | | |
|---|---|---|---|---|
| initial | Amylase | Glucoamylase | Debranching | Ethanol mg/ml, |
| moisture | units/gram | units/gram | Units/gram | 60 hours |
| 44% | 64 | 467 | 320 | 6.9 |
| 48% | 68 | 488 | 352 | 7.1 |
| 52% | 62 | 392 | 332 | 7.4 |

Final ethanol concentration in the standardized hydrolysis and fermentation represents greater than 95% of theoretical conversion of barley starch to ethanol.

### Example 5

### Process to Control Temperature in Solid Substrate Culture for Production of ATSH Enzyme

### Control of Culture Temperature by Controlling Atmosphere to Limit Metabolic Rate

The raw starch active amylase complex is produced in solid substrate culture in three steps: 1) Preparation of inoculum culture, 2) preparation and inoculation of substrate and 3) culture incubation. The following is an example of a typical enzyme production batch.

Preparation of inoculum culture Aspergillus phoenicis ATCC 15556 conditioned according to the methods of Example 1 was stored on acid barley agar slants at 4 C. Barley agar is made of 5% finely ground barley in water, 15 grams per liter agar, pH adjusted to 3.8 to 4.0 with addition of sulfuric acid, autoclave at 121° C. 15 psi and dispensed to slant tubes or petri dishes. A. phoenicis was grown on plates or slants for 72 to 96 hours until well sporulated and then stored at 4° C. for use as working cultures for inoculum preparation.

Liquid inoculum media was made up with 5% molasses and 2.5 g/l KH₂PO₄ in water and autoclaved at 121° C. 15 psi. In larger vessels media was steam sterilized in place.

A slant culture of A. phoenicis was washed with 10 ml of sterile water to suspend spores. The spore suspension was transferred to 1 liter of molasses broth and incubated on a rotary shaking water bath at 35° C. for 48 hours. The hyphal culture produced under these conditions provided the inoculum for solid substrate.

Substrate Preparation 10 kg dehulled barley flakes were mixed with 9 liters of a solution containing NH₄H₂PO₄ 13 g/l; Urea 17 g/l and 1 molar H₂SO₄ 13 ml/liter mixed to allow the barley to absorb the liquid then steamed with mixing for 30 minute to 95° C. in a paddle mixer fitted with a steam injection port. Substrate was cooled to 35° C. and inoculated at the rate of 1 liter broth culture to 10 kg dry weight substrate. Inoculated substrate was transferred to screen bottom trays to a depth of about 10 cm. Final moisture content of substrate was 50% w/w.

Culture Incubation Trays were transferred to an incubation chamber consisting of a rectangular structure fitted with air circulation, humidity sensors, oxygen sensor and temperature monitoring in air and in the culture bed. Initial conditions were air and culture bed temperature of 35 C, 80% RH, air flow rate to change chamber volume once per hour. After about 18 hours bed temperature increased to 36° C. and nitrogen gas was added to the air flow to reduce the oxygen concentration in air to 5%. Air flow and nitrogen addition was controlled in response to bed temperature to maintain temperature between 35 and 38° C. After 72 hours incubation the culture was removed from the incubation chamber and dried in a high volume flow of dry air at 40° C.

Enzyme assay Whole dried culture was assayed for raw starch activity in a standardized simultaneous hydrolysis and fermentation. The standard hydrolysis and fermentation assays contained: 25 grams ground whole barley, 2.0 ml of a freshly grown yeast culture, 1.5 ml one molar sulfuric acid to adjust pH to about 3.6, and 0.1 to 1.0 grams of dry weight equivalent enzyme preparation (typically 0.5 grams or a 2% enzyme dose) and water to 100 grams total weight of fermentation. Assays were conducted in 250 ml shake flasks incubated on a rotary shaker at 35° C. Fermentations were sampled at 24, 48 and 60 hours and assayed for ethanol concentration by gas chromatography. Fermentations contained 7.0% w/w ethanol at 48 hours.

### Example 6

### Production of Ethanol from Raw Uncooked Barley Grain Mash

This example describes production of ethanol from whole ground uncooked barley in a simultaneous hydrolysis and fermentation using amylase preparations produced in solid substrate culture.

Enzyme was produced according to the methods described in Example 2. Fermentation was as follows:

| | | |
|---|---|---|
| Ground barley | 1,500 | grams |
| Enzyme | 60 | grams |
| 1 molar H2SO4 | 130 | ml |
| Yeast inoculum | 120 | ml |
| Water | 4,190 | ml |
| Total Weight | 6,000 | grams |

Ingredients were mixed at room temperature in a 7 liter vessel with stirring, incubated at 35° C. and sampled at intervals for ethanol assay. Initial pH was 3.55 and final pH was 3.70. Agitation was for two minutes every hour. Ethanol assay was by gas chromatography. Final ethanol yield was greater than 95% of theoretical based on barley at 54% starch content.

| Fermentation Time Hours | Ethanol Concentration % w/v |
|---|---|
| 14 | 2.3 |
| 24 | 4.7 |
| 48 | 7.3 |
| 60 | 7.8 |

### Example 7

### ATSH Process for Ethanol Production from Corn

This example describes production of ethanol from whole ground uncooked corn in a simultaneous hydrolysis and fermentation using amylase preparations produced in solid substrate culture.

ATSH Enzyme was produced according to the methods described in Example 2. This example compared simultaneous hydrolysis and fermentation using two different dose rates of ATSH enzyme preparation, 4% w/w and 1% w/w based on the weight of whole ground corn used in the fermentation. Difference in weight between fermentations with 4% and 1% enzyme dose was made up with water.

Fermentations were as follows:

| | |
|---|---|
| 4% enzyme dose | 1% enzyme dose |

| | | | | |
|---|---|---|---|---|
| Ground corn | 1,500 | grams | 1,500 | grams |
| Enzyme | 60 | grams | 15 | grams |
| 1 molar H2SO4 | 90 | ml | 90 | ml |
| Yeast inoculum | 120 | ml | 120 | ml |
| Water | 4,230 | ml | 4,275 | ml |
| Total Weight | 6,000 | grams | 6,000 | grams |
| Total volume | 6,600 | ml | 6,600 | ml |

Ingredients were mixed at room temperature in a 7 liter vessel with stirring, incubated at 35° C. and sampled at intervals for ethanol assay. Initial pH was 3.3, final pH 3.7. Agitation was for two minutes every hour. Ethanol assay was by gas chromatography. Final ethanol yield was greater than 90% of theoretical based on corn at 60% starch content.

Ethanol Concentration % w/v

| Fermentation Time Hours | 4% enzyme | 1% enzyme |
|---|---|---|
| 12 | 2.0 | 1.6 |
| 24 | 3.3 | 3.6 |
| 32 | 4.8 | 4.9 |
| 48 | 6.2 | 6.1 |
| 60 | 7.1 | 6.9 |

### Example 8

### ATSH Process for Production of Ethanol from Wheat

This example describes production of ethanol from whole ground uncooked wheat in a simultaneous hydrolysis and fermentation using amylase preparations produced in solid substrate culture.

ATSH Enzyme was produced according to the methods described in Example 2. This example used simultaneous hydrolysis and fermentation using a dose rates of ATSH enzyme preparation, of 3.75% w/w based on the weight of whole ground wheat used in the fermentation. Fermentation was as follows:

| | | |
|---|---|---|
| Ground wheat | 1,200 | grams |
| Enzyme | 45 | grams |
| 1 molar H2SO4 | 60 | ml |
| Yeast inoculum | 120 | ml |
| Water | 4,515 | ml |
| Total Weight | 5940 | grams |

Ingredients were mixed at room temperature in a 7 liter vessel with stirring, incubated at 35° C. and sampled at intervals for ethanol assay. Initial pH was 3.75, final pH 3.75. Agitation was for two minutes every hour. Ethanol assay was by gas chromatography.

| Fermentation Time Hours | Ethanol Concentration % w/v |
|---|---|
| 26 | 2.4 |
| 40 | 2.7 |
| 48 | 3.6 |
| 62 | 4.1 |
| 72 | 4.3 |

### Example 9

### Sequential Hydrolysis and Fermentation

The ATSH enzyme preparation can be used in sequential hydrolysis and fermentation as well as in simultaneous hydrolysis and fermentation as described in previous examples. This example describes hydrolysis of uncooked granular starch at 35° C. followed by addition of yeast to initiate fermentation. Hydrolysis proceeded for 12 hours until about 30% of available starch was hydrolyzed to glucose. Yeast was then added to initiate fermentation.

This experiment was conducted in a 200 liter stirred tank. Ground barley was mixed with water to 25% w/w solids loading, mixed and pH adjusted to 3.5 with addition of sulfuric acid. ATSH enzyme prepared according to the method of Example 2 was added at the rate of 4% (w/w) of ground barley. The mixture was stirred and incubated at 35° C. for 12 hours at which point 500 ml of fresh yeast inoculum was added. The hydrolysis and fermentation were monitored by sampling and assayed for glucose and ethanol concentration at intervals shown in the following table. Final mash pH was 3.7. Glucose was determined using a Yellow Springs Instrument enzymatic glucose analyzer. The assay is specific for glucose. Ethanol was determined by gas chromatography. Results are shown in Table 4 below.

TABLE 4

| Time hours | Glucose g/l | Ethanol g/l |
|---|---|---|
| 5 | 24 | — |
| 8 | 29 | — |
| 12 | 33 | — |
| 24 | 58 | 33 |
| 30 | 64 | 38 |
| 36 | — | 42 |
| 48 | — | 57 |
| 72 | — | 73 |

What is claimed is:

1. A composition comprising the Aspergillus phoenicis strain deposited as NRRL 50090.

2. The composition according to claim 1, further comprising a solid state substrate.

3. The composition according to claim 2, wherein said substrate comprises barley.

4. An enzyme composition comprising an alpha amylase derived from the Aspergillus phoenicis strain deposited as NRRL 50090.

5. The enzyme composition according to claim 4, further comprising a glucoamylase and a beta glucanase.

6. A method for adapting a fungus to produce amylase that is optimal to hydrolyze starch at a low pH, comprising:
(a) inoculating a parent fungus strain in a culture medium having a first pH value;
(b) selecting a first progeny strain that is adapted for growth at said first pH value;
(c) inoculating said first progeny strain in a culture medium having a second pH value, wherein said second pH value is lower than said first pH value;
(d) selecting a second progeny strain that is adapted for growth at said second pH value;
(e) optionally repeating steps (c) and (d) till said second pH value is a final pH value; and
(f) determining if the strain obtained in step (e) is capable of producing an enzyme composition that will hydrolyze starch at a pH lower than 5.

7. The method according to claim 6, wherein said final pH value is about 3.6.

8. The method according to claim 6, wherein said first pH value is about 6.

9. The method according to claim 6, wherein said second pH value is 0.5 lower than said first pH value.

10. A method of making an enzyme composition, comprising:
(a) providing a solid state substrate;
(b) growing the Aspergillus phoenicis strain deposited as NRRL 50090 on said substrate for a first period of time in a growth chamber to produce an enzyme composition.

11. The method according to claim 10, wherein said substrate comprises barley.

12. The method according to claim 10, further comprising harvesting said enzyme composition.

13. The method according to claim 10, wherein said substrate has undergone heat treatment.

14. The method according to claim 13, wherein said heat treatment is steaming.

15. The method according to claim 14, wherein said steaming is conducted at ambient pressure.

16. The method according to claim 10, wherein said substrate has undergone radiation treatment.

17. The method according to claim 16, wherein said radiation treatment is gamma ray treatment.

18. The method according to claim 10, wherein said first period of time is four days.

19. The method according to claim 10, wherein said enzyme composition comprises an alpha amylase, a glucoamylase, and a beta glucanase.

20. The method according to claim 10, wherein said growing step comprises supplying a gas selected from the group consisting of nitrogen and carbon dioxide to the atmosphere of said growth chamber.

21. The method according to claim 10, wherein said substrate further comprises NH₄H₂PO₄. Urea, and H₂SO₄.

22. A method of producing ethanol, comprising:
(1) providing a mash that is adjusted to about pH 3.5 to 4.0;
(2) mixing said mash with (i) an enzyme composition produced with the Aspergillus phoenicis strain deposited as NRRL 50090, and (ii) a yeast; and
(3) incubating for a period of fermentation time under a temperature between 20 to 40° C. to produce ethanol.

23. The method according to claim 22, wherein said method further comprises collecting said ethanol.

24. The method according to claim 23, further comprises distilling said ethanol.

25. The method according to claim 22, wherein said mash has up to 40% (w/w) solid.

26. The method according to claim 22, wherein said fermentation time is from 26 to 72 hours.

27. The method according to claim 22, wherein said temperature is about 35° C.

28. The method according to claim 22, wherein said mash is un-gelatinized.

29. The method according to claim 22, wherein said mash is gelatinized.

30. The method according to claim 22, wherein said ethanol produced is less than 15% (w/w).
